(19) United States
(12) Patent Application Publication
(10) Pub. No.: US 2019/0079014 A1
(43) Pub. Date: Mar. 14, 2019

(54) CORE-SHELL PLASMONIC NANOGAPPED NANOSTRUCTURED MATERIAL

(71) Applicant: Nanyang Technological University, Singapore (SG)

(72) Inventors: Hongwei Duan, Singapore (SG); Jiajing Zhou, Singapore (SG)

(21) Appl. No.: 16/084,526

(22) PCT Filed: Mar. 24, 2017
(86) PCT No.: PCT/SG2017/050151

§ 371 Date: Sep. 12, 2018

(30) Foreign Application Priority Data
Mar. 24, 2016 (SG) 10201602345W

(51) Int. Cl. G01N 21/65; A61K 41/00; A61K 49/00; A61K 9/50; B01J 13/14; B01J 13/22; C09B 67/02; G01N 21/64

(52) U.S. Cl. CPC G01N 21/658; A61K 41/0052; A61K 49/0043; A61K 49/0067; A61K 9/5094; B82Y 30/00; B01J 13/22; C09B 67/0097; G01N 21/648; G01N 21/6428; B01J 13/14

(57) ABSTRACT

A core-shell plasmonic nanogapped nanostructured material is provided. The core-shell nanogapped nanostructured material has a core and at least one shell surrounding the core, wherein the at least one shell comprises a first layer comprising a polymer having a catechol group, wherein the first layer defines the nanogap in the core-shell plasmonic nanostructured material, and a second layer comprises a metal disposed on the first layer. A method of preparing the core-shell plasmonic nanogap nanostructured material, and use of the core-shell plasmonic nanogap nanostructured material are also provided. As an embodiment, a polydopamine covalently bonded to a Raman probe or a fluorescent probe is used to prepare the first layer of the shell in said core-shell plasmonic nanogap nanostructured material, thereafter a gold shell is deposited onto the polydopamine to form a second layer of the shell. In present invention, it is demonstrated that the method is highly versatile and can be used for different core materials, including magnetic Fe₃O₄ nanoparticles and metal-organic frameworks (MOF) nanoparticles. The potential application of said core-shell plasmonic nanogapped nanostructured in sensing and theranostics is also demonstrated.

### CROSS-REFERENCE TO RELATED APPLICATION

This application claims the benefit of priority of Singapore patent application No. 10201602345W filed on 24 Mar. 2016, the content of which is incorporated herein by reference in its entirety for all purposes.

### TECHNICAL FIELD

Various embodiments refer to a core-shell plasmonic nanostructured material, a method for preparing the core-shell plasmonic nanostructured material, and use of the core-shell plasmonic nanostructured material in sensing, optoelectronics or theranostics.

### BACKGROUND

Localized surface plasmons (LSPs) may arise as a result of the confinement of surface plasmons in a nanoparticle having a size that is comparable to or smaller than the wavelength of electromagnetic radiation that is used to excite the plasmons. The localized surface plasmons may have a resonant frequency at which the absorption and scattering of light occur most efficiently, which may in turn depend upon the metal and the nature of the surface such as size, roughness, shape, interparticle spacing, and dielectric environment.

The unique optical properties of plasmonic nanomaterials, originating from localized surface plasmon resonance (LSPR), are of tremendous potential across many disciplines spanning chemistry, materials science, photonics, and medicine. Development of plasmonic nanostructures with precisely controlled spectroscopic properties and/or multifunctional characteristics is key to their use in diverse applications. In particular, tailored LSPR of plasmonic nanostructures allows for spatially confining photons at sub-wavelength scales and controlling light-molecule interactions at specific wavelengths, forming the fundamental basis of their functions in surface enhanced spectroscopy and optoelectronics.

The promise of multifunctional nanoparticles, in which structurally integrated plasmonic materials and complementary counterparts lead to synergistic properties, is evident from recent progress in emerging fields such as sensing, theranostic nanomedicine, and plasmon-enhanced photochemical reactions such as photocatalysis and solar energy conversion. The strong dependence of LSPR wavelength on interparticle coupling of plasmonic nanostructures has stimulated widespread interest in nanoparticle assemblies with defined nanogaps between the building blocks.

Core-shell nanogapped nanoparticles (NNPs), or nanomatryoshkas, with a built-in dielectric gap separating the core and shell have emerged as a class of internally coupled plasmonic nanostructures. The nanogap size plays a key role in tailoring the plasmonic coupling of core and shell toward broadly tunable LSPR across visible and near-infrared (NIR) spectral range. Considerable efforts have been made in engineering the nanogap in terms of both nanogap sizes and optical encoding, using materials such as silica, DNA, and small molecules as dielectric spacers. However, it remains challenging to simultaneously achieve tailored nanogap engineering and structural integration toward multifunctional NNPs.

In view of the above, there exists a need for an improved plasmonic nanostructured material that overcomes or at least alleviates one or more of the above-mentioned problems.

### SUMMARY

In a first aspect, a core-shell plasmonic nanostructured material is provided. The core-shell plasmonic nanostructured material has a core and at least one shell surrounding the core, wherein the at least one shell comprises
a) a first layer comprising a polymer having a catechol group, the first layer defining a nanogap in the core-shell plasmonic nanostructured material, and
b) a second layer comprising a metal disposed on the first layer.

In a second aspect, a method for preparing a core-shell plasmonic nanostructured material having a core and at least one shell surrounding the core is provided. The method comprises
a) providing a nanostructured material, and
b) forming at least one shell on the nanostructured material by
   i. forming a first layer comprising a polymer having a catechol group on the nanostructured material, the first layer defining a nanogap in the core-shell plasmonic nanostructured material, and
   ii. forming a second layer comprising a metal on the first layer.

In a third aspect, use of a core-shell plasmonic nanostructured material according to the first aspect or a core-shell plasmonic nanostructured material prepared by a method according to the second aspect in sensing, optoelectronics or theranostics is provided.

### BRIEF DESCRIPTION OF THE DRAWINGS

The invention will be better understood with reference to the detailed description when considered in conjunction with the non-limiting examples and the accompanying drawings, in which:

FIG. 1 is a schematic diagram showing synthesis of nanogapped nanoparticles (NNPs) based on polydopamine (PDA) coating according to embodiments.

FIG. 2A is a transmission electron microscopy (TEM) image of 20 nm gold (Au) nanoparticles (NPs) with a 2 nm PDA coating.

FIG. 2B is a TEM image of 20 nm Au nanoparticles with a 5 nm PDA coating.

FIG. 2C is a TEM image of 20 nm Au nanoparticles with a 13 nm PDA coating.

FIG. 2D is a TEM image of NNPs with a built-in nanogap of 2 nm.

FIG. 2E is a TEM image of NNPs with a built-in nanogap of 5 nm.

FIG. 2F is a TEM image of NNPs with a built-in nanogap of 13 nm.

FIG. 2G is a TEM image of NNPs (50 nm Au nanoparticles as cores) with a single shell.

FIG. 2H is a TEM image of NNPs (50 nm Au nanoparticles as cores) with double shells.

FIG. 2I is a TEM image of NNPs (50 nm Au nanoparticles as cores) with triple shells.

FIG. 2J is a TEM image of 50 nm Au nanoparticles.

FIG. 3A is a TEM image of 20 nm Au nanoparticles.

FIG. 3B is a TEM image of 20 nm Au nanoparticles with 2 nm thickness of PDA coating.

FIG. 3C is a TEM image of 20 nm Au nanoparticles with 5 nm thickness of PDA coating.

FIG. 3D is a TEM image of 20 nm Au nanoparticles with 13 nm thickness of PDA coating.

FIG. 4A is a TEM image of 20 nm AuNP.

FIG. 4B is a TEM image of Au@PDA-20.

FIG. 4C is a TEM image of Au@PDA-20@Au.

FIG. 4D is UV-vis spectra of AuNP, Au@PDA-20, and Au@PDA-20@Au.

FIG. 5A is a scanning electron microscopy (SEM) image of the nanogapped nanoparticle at 0 uL of Au precursor.

FIG. 5B is a SEM image of the nanogapped nanoparticle at 25 uL of Au precursor.

FIG. 5C is a SEM image of the nanogapped nanoparticle at 50 uL of Au precursor.

FIG. 5D is a SEM image of the nanogapped nanoparticle at 80 uL of Au precursor.

FIG. 6 is a TEM image of Au(50nm)@PDA.

FIG. 7A is a TEM image of Au(50nm)@PDA@Au.

FIG. 7B is a TEM image of Au(50nm)@PDA@Au@PDA.

FIG. 8A is a graph depicting UV-vis spectra of 20 nm Au nanoparticles and Au nanoparticles with PDA coating of different thickness: 0, 2, 5, and 13 nm.

FIG. 8B is a graph showing UV-vis spectra of 20 nm Au nanoparticles and NNPs with different gap size.

FIG. 8C is a graph showing UV-vis spectra of 50 nm Au nanoparticles, Au nanoparticles coated with PDA, and NNPs with different shell number.

FIG. 8D is a graph showing SERS spectra of different Au nanostructures with RhB tags positioned on PDA layer.

FIG. 9 is a graph showing UV-vis spectra of Au nanogapped nanoparticles at different stage of growth.

FIG. 10A is a schematic diagram showing PDA-coated substrates conjugated with a rhodamine B carrying amino group (H2N-RhB) via Michael addition and/or Schiff base reaction.

FIG. 10B is a schematic diagram showing PDA-coated nanoparticles conjugated with thiol and amine groups via Michael addition and/or Schiff base reaction.

FIG. 11 is a graph showing surface enhanced Raman spectroscopy (SERS) intensity at 1647 cm⁻¹ as a function of the average number of RhB molecules loaded in each Au NNP.

FIG. 12A is a graph showing SERS spectra of the as-prepared nanogapped nanoparticles dispersed in water at 0, 6, 12, and 24 h.

FIG. 12B is a graph showing time-dependent SERS intensity at 1647 cm⁻¹ over 24 h.

FIG. 13A is a TEM image of AuNR.

FIG. 13B is a TEM image of AuNR@PDA.

FIG. 13C is a TEM image of AuNR@PDA@Au.

FIG. 13D is a TEM image of UiO-66. Inset: sample at higher magnification; scale bar=100 nm.

FIG. 13E is a TEM image of UiO-66@PDA@Au (UiO-66@single shell). Inset: sample at higher magnification; scale bar=100 nm.

FIG. 13F is a TEM image of UiO-66@PDA@Au@PDA@Au (UiO-66@double shell). Inset: sample at higher magnification; scale bar=100 nm.

FIG. 14A is UV-vis spectra of plasmonic AuNR at different stages: AuNR, AuNR@PDA, and AuNR@PDA@Au.

FIG. 14B is a graph depicting UV-vis spectra of plasmonic UiO-66-cored NNPs at different stages.

FIG. 15A is a TEM image of magnetic iron oxide nanoparticles@PDA@Au (MagNP@PDA@Au).

FIG. 15B is a TEM image of magnetic NNPs. Inset: Photo of magnetic separation of the magnetic NNPs.

FIG. 15C is a graph depicting UV-vis spectra of magnetic NNPs at different stages of growth.

FIG. 15D is a graph showing SERS spectra of NTP-encoded magnetic NNPs and the control nanoparticles.

FIG. 16A is a TEM image of MagNP.

FIG. 16B is a TEM image of MagNP@PDA.

FIG. 17A is a schematic diagram of the immunoassay using SERS-encoded magnetic NNPs for bacterial detection.

FIG. 17B is a graph showing SERS spectra of different concentrations of E. coli O157:H7. Inset: Raman intensity at 1341 cm⁻¹ vs logarithm of the corresponding E. coli O157:H7 concentration.

FIG. 17C is a graph showing Raman intensity at 1341 cm⁻¹ of the substrates from the assays of control buffers and various types of bacteria (10⁶ CFU/mL).

FIG. 18A is a SEM image of E. coli O157:H7 before capture by magnetic NNPs.

FIG. 18B is a SEM image of E. coli O157:H7 after capture by magnetic NNPs.

FIG. 19 is a graph depicting photothermal conversion of magnetic NNPs exposed to an 808 nm laser (1 W/cm²) at OD₈₀₈ₙₘ=1.5.

FIG. 20A is a fluorescence image of the captured bacteria before photothermal treatment by exposure to an 808 nm laser (1 W/cm²) for 15 min.

FIG. 20B is a fluorescence image of the captured bacteria after photothermal treatment by exposure to an 808 nm laser (1 W/cm²) for 15 min.

FIG. 21A is a graph showing UV-vis spectra of Au@PDA and Au@PDA@Ag nanomatryoshkas.

FIG. 21B is a TEM image of Au@PDA@Ag nanomatryoshkas.

FIG. 22A is a TEM image of Au@Eccentric PDA. Eccentric is relative to concentric. In the case of eccentric PDA coating, the Au core is not right in the center, which would induce some interesting optical properties.

FIG. 22B is a TEM image of Au@Eccentric PDA@Au. Eccentric is relative to concentric. In the case of eccentric PDA coating, the Au core is not right in the center, which would induce some interesting optical properties.

FIG. 23 is a graph depicting photothermal conversion of different Au nanostructures exposed to an 808 nm laser (1 W/cm²).

FIG. 24 is a graph showing SERS spectra of different nanogapped Au nanostructures (Au NNPs) without Raman dyes.

FIG. 25A is a TEM image of MagNP@PDA@Au@PDA at low magnification.

FIG. 25B is a TEM image of MagNP@PDA@Au@PDA at high magnification.

FIG. 25C is a TEM image of MagNP@PDA@Au@PDA@Au at high magnification.

### DETAILED DESCRIPTION

Various embodiments refer in a first aspect to a core-shell plasmonic nanostructured material having a core and at least one shell surrounding the core. The at least one shell comprises a first layer comprising a polymer having a catechol group, wherein the first layer defines a nanogap in the core-shell plasmonic nanostructured material, and a second layer comprising a metal disposed on the first layer.

The polymer having a catechol group may, for example, be a catecholamine-based polymer such as polydopamine. Advantageously, polymer having a catechol group such as polydopamine is able to adhere to virtually any solid substrate, and form a rigid conformal coating with controlled thickness in the nanometer scale by depositing from an aqueous solution. Size of the nanogap defined by the first layer may therefore be controlled easily to vary plasmonic coupling of the core and the shell. The polymers' adhesion abilities also mean that multiple shells may be formed around a diverse range of nanostructured materials, such as inorganic, organic, or hybrid functional cores of different sizes, shapes, and chemical compositions. At the same time, high density of catechol groups on the polymers may impart reducing activity to the polymer, facilitating in-situ nucleation and deposition of a metallic layer thereon. The nanogap may furthermore act as an electromagnetic hot-spot, such that by positioning molecular probes in the nanogap, for example, amplified optical signals for surface enhanced Raman scattering (SERS) may be generated.

The term “nanostructured material” as used herein refers to a material having a size measured in nanometers (nm), as well as a material having a structural feature with a size measured in nanometers. The size measured in nanometers may, for example, be less than 100 nm. The term “core-shell nanostructured material” refers to a structural configuration of a nanostructured material in which an external layer formed of a second material encompasses an inner core of a first material, thereby forming the core-shell structure. In various embodiments, the shell completely encompasses or encapsulates the core.

The core-shell plasmonic nanostructured material disclosed herein may have a regular shape such as a nanosphere, a nanorod, or be irregularly shaped, and size of the core-shell plasmonic nanostructured material may be characterized by its diameter. The term “diameter” as used herein refers to the maximal length of a straight line segment passing through the center of a figure and terminating at the periphery. Although the term “diameter” is used normally to refer to the maximal length of a line segment passing through the centre and connecting two points on the periphery of a nanosphere, it is also used herein to refer to the maximal length of a line segment passing through the centre and connecting two points on the periphery of a nanostructured material having other shapes, such as a nanorod, a nanocube or a irregularly shaped nanoparticle.

As mentioned above, a nanostructured material referred to herein may have a structural feature with a size measured in nanometers. In various embodiments, the structural feature has a size of less than 100 nm. With this in mind, a core-shell plasmonic nanostructured material as presently disclosed may include a core of a first material having a diameter that is greater than or less than 100 nm, while having an external layer formed of a second material having a thickness of less than 100 nm. As another example, a core-shell plasmonic nanostructured material may include a core of a first material having a diameter that is less than 100 nm, while having an external layer formed of a second material having a thickness of greater than or less than 100 nm.

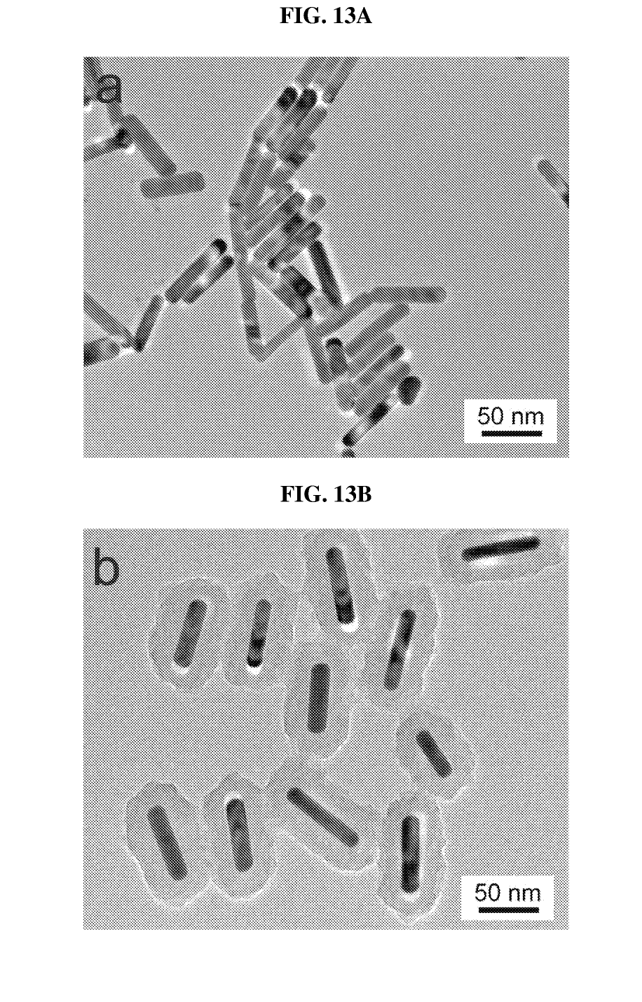

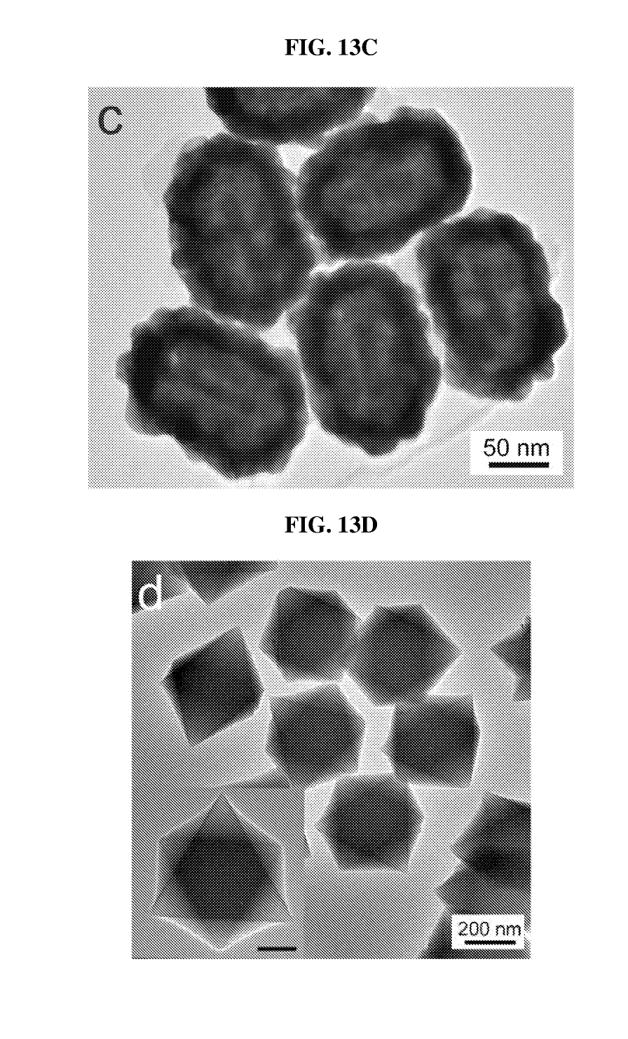

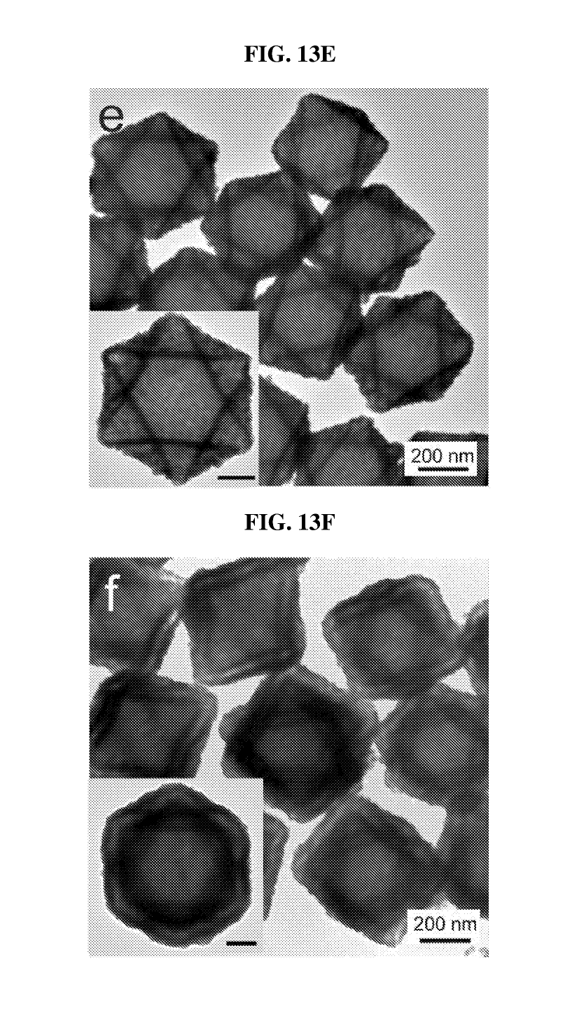

As shown in FIG. 13F, for example, a core-shell plasmonic nanostructured material according to an embodiment may have a core having a diameter of about 300 nm and a shell comprising a first layer defining a nanogap of about 50 nm in thickness, and a second layer comprising a metal having a thickness of about 30 nm disposed on the first layer.

As mentioned above, nanostructured materials of different sizes, shapes, and chemical compositions may form the core of the core-shell plasmonic nanostructured material disclosed herein. Generally, any material upon which the polymer having a catechol group may adhere may be used as to form the core. In various embodiments, the core comprises a material selected from the group consisting of a metal, a metal oxide, a metal-organic framework, a polymer, a magnetic material, a fluorescent quantum dot, and combinations thereof. For example, the core may comprise a material selected from the group consisting of gold, UiO-66, polystyrene-trapped magnetic iron oxide, and combinations thereof.

In some embodiments, the core is formed of a magnetic material. The magnetic material may, for example, be a nanoparticle having a core-shell structure. For example, the core of the magnetic particle may comprise a magnetic material, such as a ferromagnetic material and/or a superparamagnetic material. As used herein, the term “ferromagnetic” refers to a material which may be magnetized by applying an external magnetic field, and which is able to exhibit remnant magnetization upon removal of the external magnetic field. The ferromagnetic material may, for example, be attracted by a magnetic field. Examples of a ferromagnetic material include a ferromagnetic metal such as Fe, Co, Ni, FeAu, FePt, FePd, and/or CoPt, a ferromagnetic metal oxide such as Fe₂O₃, Fe₃O₄, CoO, NiO, CoFe₂O₄, and/or MnFe₂O₄, a heterogeneous structure comprising a ferromagnetic metal and/or a ferromagnetic metal oxide such as Au—Fe₂O₃, Ag—Fe₃O₄, quantum dot-Fe₂O₃ structure, or combinations of the afore-mentioned.

The term “superparamagnetic”, on the other hand, refers to a class of material that has a similar magnetism as ferromagnetic materials in the external magnetic field, but does not have a remnant magnetization after removal of the external magnetic field. In other words, a superparamagnetic material may be a material which may be magnetized by applying an external magnetic field, and which does not exhibit magnetization upon removal of the external magnetic field.

A ferromagnetic material may become superparamagnetic when the ferromagnetic material is reduced to a certain size/dimension. The threshold at which a ferromagnetic material becomes superparamagnetic may, for example, depend on the composition of the material and its size. In this regard, a person skilled in the art is able to determine when a ferromagnetic material of a specific composition and/or size becomes superparamagnetic.

Examples of a superparamagnetic material include a superparamagnetic metal, a superparamagnetic metal oxide, a heterogeneous structure comprising a superparamagnetic metal and/or a superparamagnetic metal oxide, or combinations of the afore-mentioned.

The shell of the magnetic material may comprise any suitable material that is able to form a shell surrounding the core of the magnetic particle, such as a polymer, silica, a metal, a metal-organic framework comprising compounds formed of metal ions or metal clusters coordinated to organic molecules to form one-, two-, or three-dimensional structures, or combinations thereof. Advantageously, the shell, for example, polymer or silica shell, may help to protect the magnetic core by keeping it intact and stable from outer harsh environment, such as an acid environment. In various embodiments, the polymer is selected from the group consisting of polystyrene, polymethacrylate, phenol formaldehyde resin, copolymers thereof, and combinations thereof. In specific embodiments, the polymer comprises or consists of polystyrene.

The magnetic particle having a core-shell structure may be prepared using a miniemulsion polymerization process. For example, an initiator such as potassium peroxydisulfate (KPS), azodiisobutyronitrile, or benzoyl peroxide may be added to a first liquid reagent comprising particles of a magnetic material dispersed in an aqueous solution, and stirred to dissolve the initiator in the first liquid reagent. A second liquid reagent containing monomers may be added to the resultant mixture, whereby the monomers undergo polymerization to allow formation of a polymer as a shell surrounding the particles to obtain the magnetic core-shell particles. In some embodiments, the magnetic particle has a core-shell structure, the core comprising a superparamagnetic metal oxide such as Fe₂O₃, Fe₃O₄, CoO, NiO, CoFe₂O₄, and/or MnFe₂O₄, and the shell comprising a polymer surrounding the core. In specific embodiments, the magnetic particle has a core-shell structure, the core comprising Fe₃O₄ and the shell comprising polystyrene surrounding the core.

Shape of the core of the core-shell plasmonic nanostructured material is not particularly limited and may for example, be a nanoparticle, a nanocube, a nanosphere, or a nanorod. In some embodiments, the core is a nanoparticle or a nanorod. Size of the core of the core-shell plasmonic nanostructured material is also not particularly limited, and may be of any suitable size as defined above.

At least one shell may surround the core. The at least one shell may comprise a first layer comprising a polymer having a catechol group, wherein the first layer defines a nanogap in the core-shell plasmonic nanostructured material, and a second layer comprising a metal disposed on the first layer. Accordingly, the core-shell nanostructured material disclosed herein may be termed a core-shell plasmonic nanostructured material, wherein the term “plasmonic” refers to an effect or condition involving or relating to the collective oscillation of conduction-band electrons in a medium in response to an electromagnetic radiation. As disclosed herein, the plasmonic effects exhibited by the core-shell plasmonic nanostructured material may be generated from a nanogap defined by a first layer comprised in the shell of the core-shell plasmonic nanostructured material. The term “nanogap” refers generally to a nanometric gap formed by a pair of electrically conductive surfaces, such as between the core and the second layer of the shell of the core-shell plasmonic nanostructured material. Even though nanogaps which have dimensions in the nanoscale are generally deemed to be extremely difficult to modulate, it has been demonstrated using a method disclosed herein that size of the nanogap defined by the first layer, for example, may be controlled easily to vary plasmonic coupling of the core and the shell.

The first layer of the shell may comprise or consist of a polymer having a catechol group. In various embodiments, the polymer having a catechol group is a catecholamine-based polymer. In some embodiments, the polymer having a catechol group is selected from the group consisting of polydopamine, poly(norepinephrine), poly(L-3,4-dihydroxyphenylalanine), poly(5,6-dihydroxyl-1H-benzimidazole), polyphenol, dopamine-modified poly(L-glutamic acid), dopamine-modified polyphenol, dopamine-modified poly(ethyleneimine), polydopamine and Cu²⁺, polyphenol and Fe³⁺, copolymers thereof, and combinations thereof.

In specific embodiments, the polymer having a catechol group comprises polydopamine. Polydopamine refers to a polymer obtainable by polymerization of dopamine, which refers to a chemical compound having the following formula:

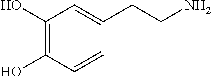

In various embodiments, the first layer comprising the polymer having a catechol group is a continuous conformal coating of the polymer disposed on the core. In some embodiments, the first layer comprising the polymer having a catechol group is in direct contact with the core.

The first layer comprising the polymer having a catechol group may have a thickness of at least 2 nm, such as about 2 nm to about 50 nm, about 2 nm to about 20 nm, about 10 nm to about 20 nm, about 15 nm to about 20 nm, about 2 nm to about 15 nm, about 2 nm to about 10 nm, about 2 nm to about 5 nm, or about 8 nm to about 12 nm. While a single deposition of the polymer may result in a thickness in the range of about 2 nm to about 13 nm depending on the type of process used, polymer with a larger thickness may be achieved by carrying out multiple depositions of the polymer. For example, a polymer thickness of about 20 nm may be achieved by carrying out the deposition process twice. Generally, there is no upper limit as to thickness of the first layer, and thickness of the first layer may be varied or controlled by, for example, varying the number of deposition cycles as mentioned above. However, nanogaps with metal-enhanced properties, such as in the form of SERS or metal-enhanced fluorescence, have usually a thickness in the range of about 2 nm to about 50 nm.

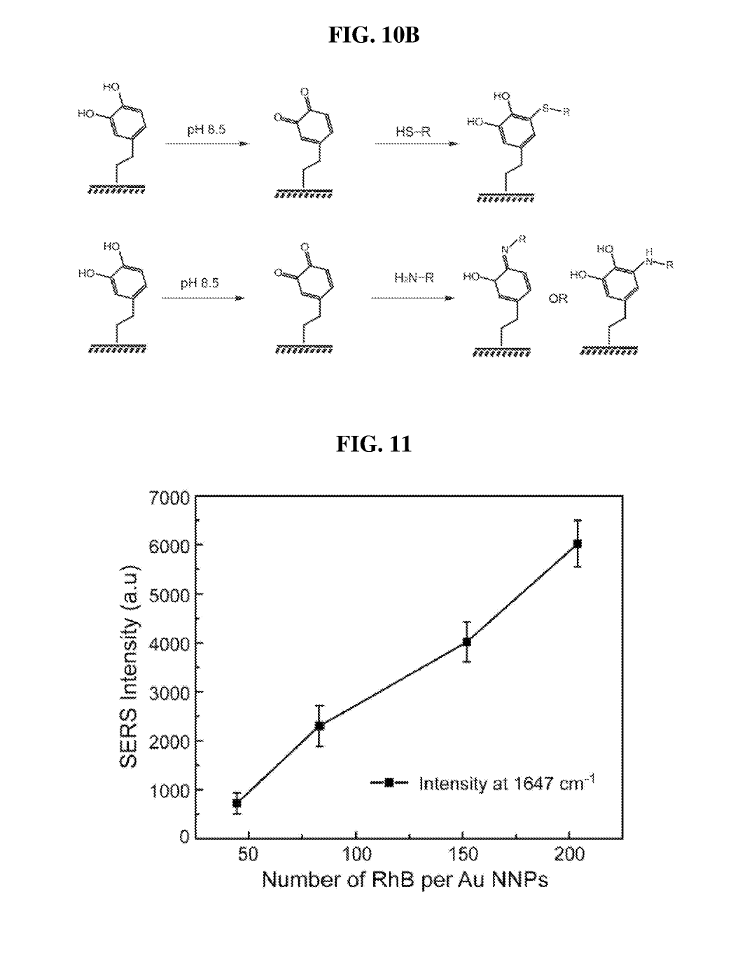

In some embodiments, the first layer further comprises a signal probe. The signal probe may, for example, be at least one of a Raman probe or a fluorescent probe. The Raman probe may be selected from the group consisting of rhodamine B, rhodamine 6G, 4-nitrothiolphenol, 4-bromothiophenol, 3,5-difluorothiophenol, and combinations thereof. The fluorescent probe, on the other hand, may be selected from the group consisting of fluorescein, rhodamine 6G, 2′,7′-dichlorodihydrofluorescein, and combinations thereof. In some embodiments, the signal probe is covalently bonded to the polymer having a catechol group comprised in the first layer. Examples are depicted in FIGS. 10A and 10B, wherein a Raman probe is covalently bonded to the polymer having a catechol group comprised in the first layer.

By securing one or more signal probes inside the nanogap, this insulates them from interfering factors from the surrounding environment, which is important for quantitative detection. Advantageously, stable, quantitative molecular fixation of the signal probes in the nanogap may translate into amplification of the signals, and in turn improve sensitivity of detection or sensing. For example, fixation of the Raman probes in the SERS-active nanogap may amplify the SERS signal and in turn, improve sensitivity of detection or sensing using SERS. Similarly, when a fluorescent probe is located in the nanogap, metal-enhanced fluorescence may be observed.

The at least one shell surrounding the core of the core-shell plasmonic nanostructured material also includes a second layer comprising or consisting of a metal disposed on the first layer. The metal may, for example, be gold, silver, aluminum, platinum, palladium, and/or copper. In various embodiments, the metal is gold.

The second layer of the shell may be a continuous conformal coating of the metal disposed on the first layer, and/or may have any suitable thickness. Thickness of the second layer of the shell may, for example, be at least 5 nm. As in the case for the first layer, it is possible to achieve a second layer having larger thickness by carrying out multiple depositions of the second layer. Accordingly, thickness of the second layer of the shell may be as thick as 100 nm or more after multiple processes. In specific embodiments, the second layer of the shell may have a thickness in the range of about 10 nm to about 100 nm, such as about 10 nm to about 50 nm, about 10 nm to about 30 nm, about 10 nm to about 20nm, or about 15 nm.

In specific embodiments, the second layer of the shell has a thickness of about 15 nm. It has been found by the inventors that this thickness gives rise to an optimal signal with highest intensity for both SERS and fluorescence. At higher thicknesses, intensity of the SERS signal or fluorescence from the nanogap may be re-absorbed by the second layer, to give rise to a decreased signal level. The first layer and the second layer comprised in the shell of the core-shell plasmonic nanostructured material may be of the same or a different thickness to each other.

In some embodiments, the second layer further comprises an analyte-binding molecule attached to the metal. This may allow modulation of the interaction of the core-shell plasmonic nanostructured material with biological systems. The analyte-binding molecules may be covalently bonded to the metal via a linker, involving use of functional groups such as thiol group, carboxy group, and/or amino group, or via ester bonding, for example. In embodiments wherein the metal is gold, for example, the analyte-binding molecule may be covalently bonded to the gold surface via a thiol group by forming thiol-Au bond. The analyte-binding molecule may, for example, be selected from the group consisting of an antibody, DNA aptamer, RNA, and combinations thereof. For example, monoclonal antibody (8B1-C2-B1) may be used to specifically bind to bacteria E. coli O157:H7, while MUC-1 aptamer may be used to specifically bind to breast cancer cell line, MCF-7.

In various embodiments, the core-shell plasmonic nanostructured material comprises two or more shells, such as two, three, four, five, six, eight, or ten shells. In some embodiments, the number of shells in the core-shell plasmonic nanostructured material comprises is two or three.

Each of the two or more shells may have a first layer comprising a polymer having a catechol group, the first layer defining a nanogap in the core-shell plasmonic nanostructured material, and a second layer comprising a metal disposed on the first layer. Examples of suitable polymer and metal, which may respectively be comprised in the first layer and the second layer, have already been discussed above.

In some embodiments, the core-shell plasmonic nanostructured material comprises two shells. The two shells may surround the core, and be formed such that the first layer of the first shell is disposed directly on the core, the second layer of the first shell is disposed directly on the first layer of the first shell; the first layer of the second shell is disposed directly on the second layer of the first shell, and the second layer of the second shell is disposed directly on the first layer of the second shell.

It follows from the above discussion that, in embodiments wherein the core-shell plasmonic nanostructured material comprises three shells, the first layer of the third shell may be disposed directly on the second layer of the second shell, and the second layer of the third shell may be disposed directly on the first layer of the third shell.

Each first layer in the two or more shells may define a nanogap in the core-shell plasmonic nanostructured material. Advantageously, by varying a thickness of each of the first layers and/or type of polymer comprised in the first layer, for example, plasmonic properties of the core-shell plasmonic nanostructured material may be tailored according to specific applications. In various embodiments, each first layer in the two or more shells may be configured such that it has at least one of (i) a different polymer having a catechol group, (ii) a different thickness, or (iii) a different signal probe when present. Likewise, each second layer in the two or more shells may comprise a different metal.

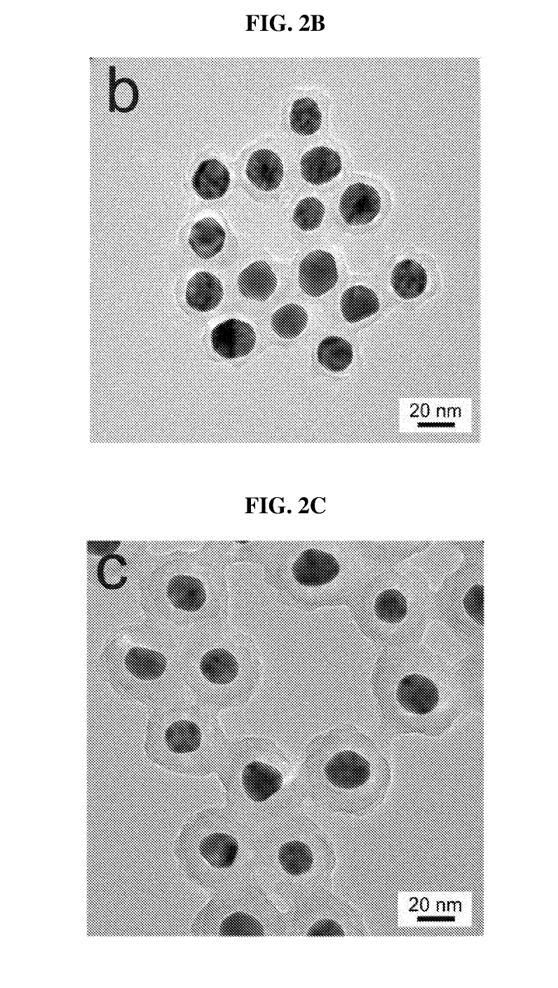

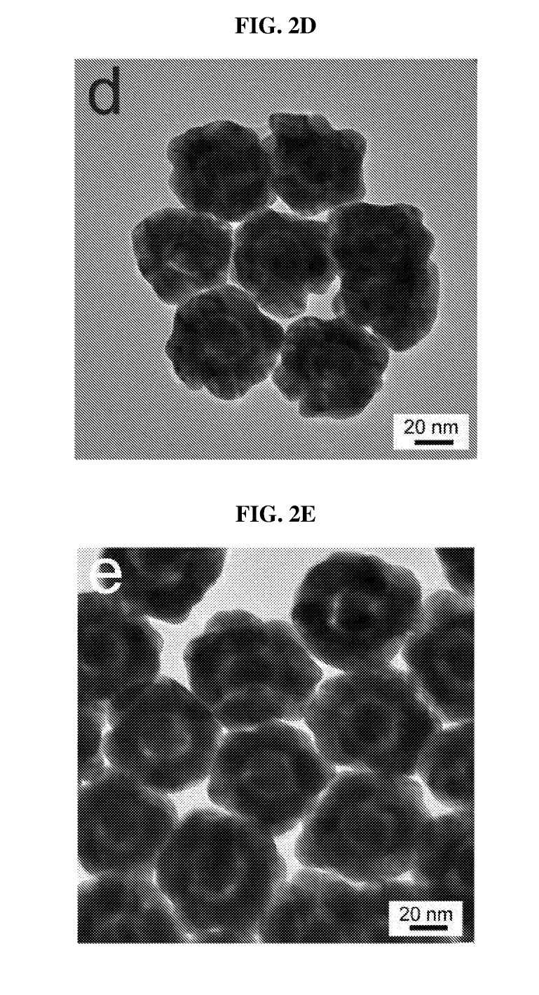

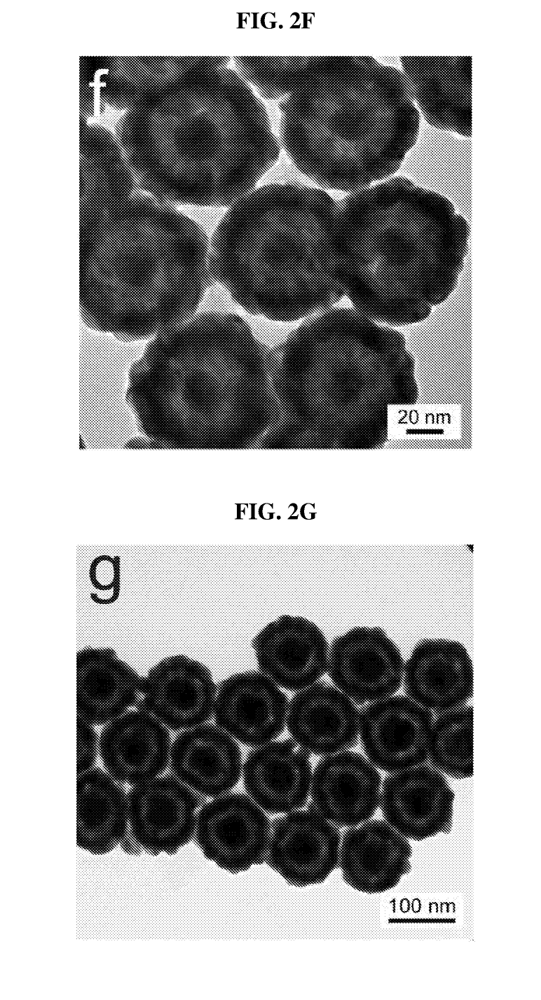

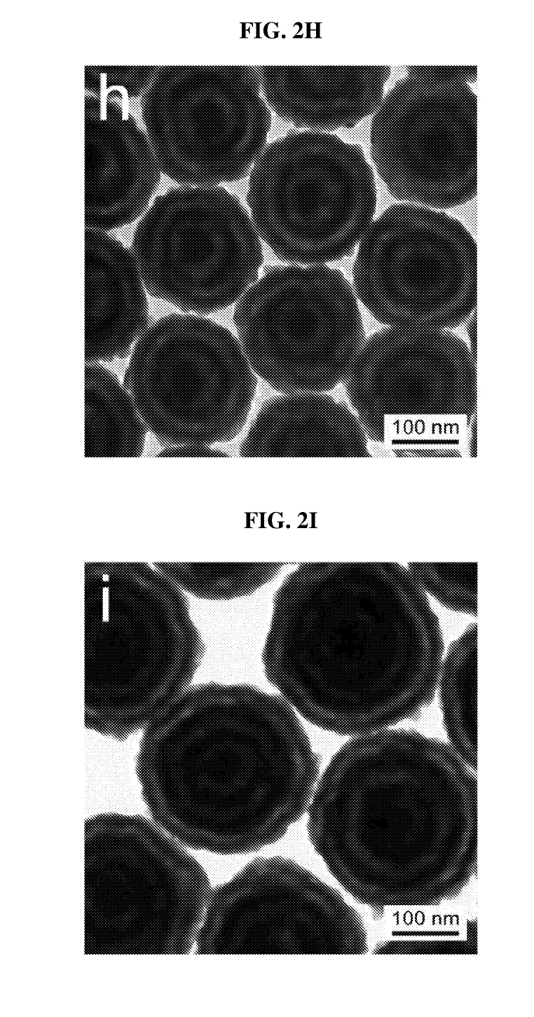

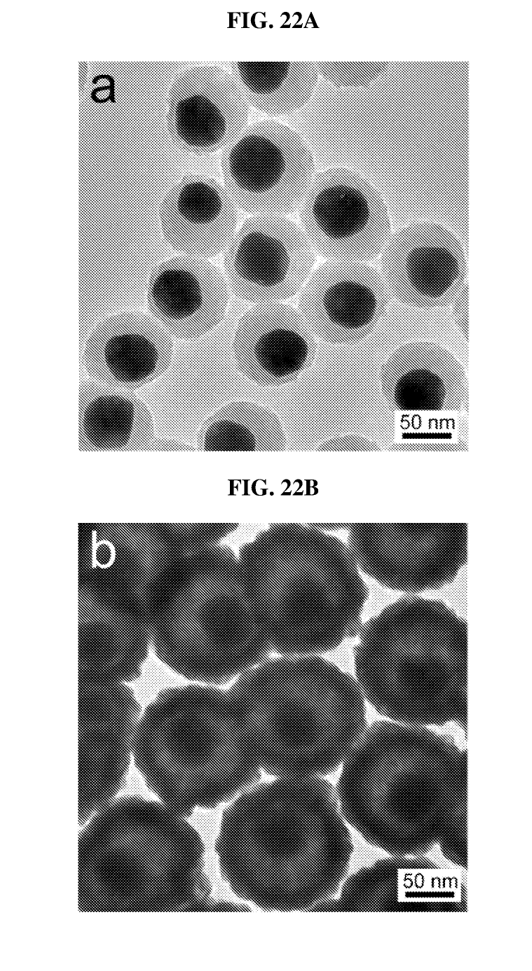

In various embodiments, the at least one shell is concentrically disposed about the core, such as that shown in FIG. 2C. In some embodiments, the at least one shell is eccentrically disposed about the core, such as that shown in FIG. 22A.

Various embodiments refer in a second aspect to a method for preparing a core-shell plasmonic nanostructured material having a core and at least one shell surrounding the core. The method may comprise providing a nanostructured material, and forming at least one shell on the nanostructured material by forming a first layer comprising a polymer having a catechol group on the nanostructured material, the first layer defining a nanogap in the core-shell plasmonic nanostructured material, and forming a second layer comprising a metal on the first layer.

Suitable nanostructured materials which may be used as the core have already been mentioned above.

In various embodiments, forming the first layer is carried out by polymerizing monomers of the polymer having a catechol group on the nanostructured material. This may take place via self-polymerization, such as in the case of polydopamine, to allow formation of a polymer having a catechol group as a shell surrounding the nanostructured material. The monomers may be comprised in an aqueous solution having a pH of about 7.1 to about 12, such as about 7.1 to about 9.0. Advantageously, alkaline conditions may induce or facilitate self-polymerization of the monomers. To achieve this, a liquid reagent such as TRIS-buffer solution, bicine buffer solution, and/or ammonia solution may be added to the aqueous solution.

The nanostructured material may be dispersed in a solution comprising the monomers using any suitable agitation methods such as stirring, shaking, agitating, and/or vortexing. Generally, a higher concentration of the monomers in solution may give rise to formation of a thicker layer of the polymer on the core. The thickness of the polymer may also depend on the size and/or the number of the cores present. For example, at the same monomer concentration, larger size of cores and/or larger number of cores may result in formation of polymer layers with lower thickness on the cores. Polymerizing the monomers on the nanostructured material may be carried out for any suitable time period that allows formation of a polymer on the nanostructured material. In various embodiments, polymerizing the monomers on the nanostructured material is carried out for a time period of 8 hours or more.

In some embodiments, forming the first layer further comprises covalently binding a signal probe to the polymer having a catechol group. Examples of suitable signal probes have already been discussed above. In some embodiments, quinone groups present in the polymer may undergo spontaneous Michael addition and/or Schiff base reactions, hence a Raman probe such as rhodamine B carrying an amino group may be conjugated to the polymer via Michael addition and/or Schiff base reaction.

The method disclosed herein comprises forming a second layer comprising a metal on the first layer. For example, a metal precursor in the form of a metal salt may be added to a suspension containing the nanostructured material having the first layer of polymer formed thereon, such that the catechol groups on the polymerized material may reduce the monovalent or the bivalent metal ions into its zerovalent state. In so doing, the metal ions may precipitate out in the reaction mixture in their metal form, to form a second layer of the metal on the first layer.

In various embodiments, the metal comprised in the second layer is gold. A gold salt including a metal gold salt, such as HAuCl₄, KAuCl₄, and/or NaAuCl₄ may be used, and forming a second layer comprising a metal on the first layer may accordingly comprise contacting the first layer with the gold salt at alkaline conditions.

By increasing amounts of the gold salt used, thickness of the metal comprised in the second layer may be increased. The layer of metal formed may comprise or consist of gold nanoparticles. The gold nanoparticles may bind to the active surface groups, such as hydroxyl (—OH) and amine (—NH₂) groups on the polymerized material, which then holds the gold nanoparticles in place to form a layer of metal on the nanostructured material.

In some embodiments, the gold salt comprises or consists of a metal gold salt, such as an alkali metal gold salt. Advantageously, use of metal gold salt or alkali metal gold salt such as KAuCl₄ and/or NaAuCl₄ avoids issues relating to use of HAuCl₄ which induces an acidic environment that causes catechol groups to have weak reducing power. This may translate into insufficient reducing power of the first layer in reducing the gold salt to form the second layer. The HAuCl₄ may also cause degradation of the polymer comprising a catechol group, thereby reducing quality of the nanogap and in turn plasmonic performance of the nanostructured material.

As mentioned above, high density of catechol groups on the polymers may impart reducing activity to the polymer, facilitating in-situ nucleation and deposition of a metallic layer thereon. A reducing agent, such as NH₂OH.HCl, ascorbic acid, and/or hydroquinone, may nevertheless be added to facilitate reduction of the metal ions in the reaction mixture in their metal form so as to form a second layer of the metal on the first layer. Choice of whether or not to include the reducing agent may depend, for example, on the metal precursor used, amount of catechol groups present in the first layer, and/or whether or not a second layer that completely encapsulates the nanostructured material having the first layer of polymer formed thereon is desired.

For example, in embodiments wherein the number of catechol groups comprised in the first layer is low due to formation of a thin first layer, for example, a reducing agent may be added to facilitate formation of a second layer that encapsulates the nanostructured material having the first layer of polymer formed thereon. As a further example, even though HAuCl₄ as mentioned above may induce an acidic environment that causes catechol groups to have weak reducing power, addition of a reducing agent may allow formation of a second layer that encapsulates the nanostructured material having the first layer of polymer formed thereon.

A reducing agent may also be added in embodiments wherein a second or multiple depositions of the second layer is carried out to increase thickness of the second layer, since multiple depositions of the second layer may mean that the second and subsequent deposition of the second layer is not carried out on the first layer comprising a polymer having catechol groups which may impart reducing activity to the polymer. For example, a metal precursor in the form of a metal salt may be added along with a reducing agent to a suspension containing the core-shell plasmonic nanostructured material, such that the reducing agent may reduce the monovalent or the bivalent metal ions into its zerovalent state. In so doing, the metal ions may precipitate out in the reaction mixture in their metal form, to form a further coating of the second layer of the metal on the core-shell plasmonic nanostructured material.

As mentioned above, the core-shell plasmonic nanostructured material may comprise two or more shells. For example, the nanostructured material forming a core of the core-shell plasmonic nanostructured material may be added into a mixture containing monomers of the polymer having a catechol group so that the nanostructured material may function as seeds onto which the polymer having a catechol group may be coated thereon. Subsequently, the second layer comprising a metal disposed on the first layer may be formed by adding the nanostructured material containing the first layer into a mixture containing a metal precursor such as a gold salt, a metal gold salt or an alkali metal gold salt as mentioned above, wherein the metal precursor may be reduced by the catechol group on the polymer to form the second layer. By repeating this process one or more times, two or more shells may be formed on the nanostructured material.

In various embodiments, forming each first layer of the two or more shells may comprise forming each first layer using at least one of (i) a different polymer having a catechol group, (ii) a different thickness, or (iii) a different signal probe when present. Examples of suitable polymers and signal probes have already been discussed above. Likewise, forming each second layer of the two or more shells may comprise forming each second layer with a different metal.

Various embodiments refer in a third aspect to use of a core-shell plasmonic nanostructured material according to the first aspect or a core-shell plasmonic nanostructured material prepared by a method according to the second aspect in sensing, optoelectronics or theranostics.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

Various embodiments relate to a new platform strategy that offers unprecedented flexibility in synthesizing plasmonic nanogapped nanoparticles (NNPs) containing a built-in nanogap, which is an intriguing type of internally coupled plasmonic nanostructures of considerable interest for a wide spectrum of applications.

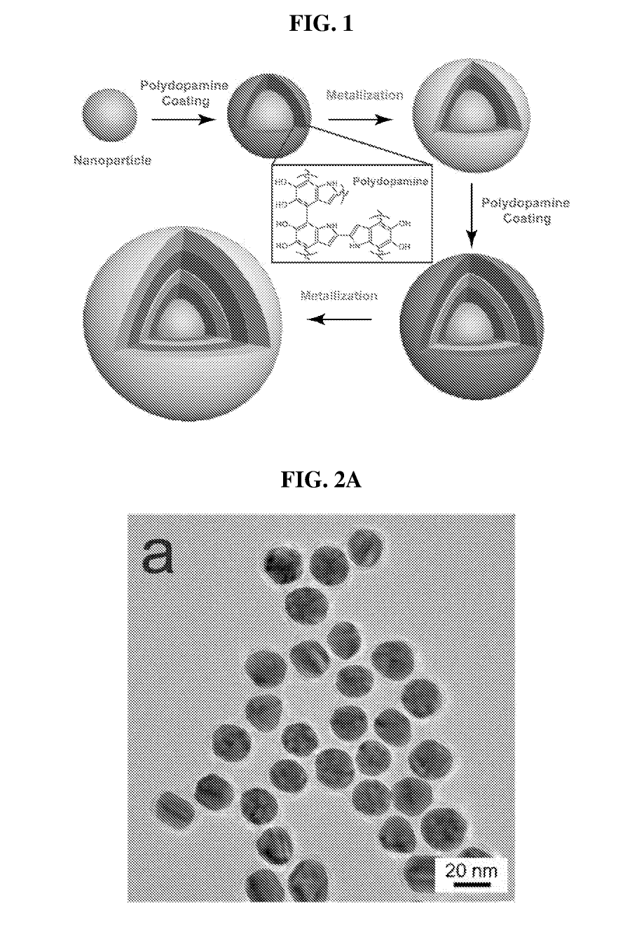

The platform strategy disclosed herein is based on the use of mussel-inspired polydopamine (PDA) to realize unprecedented flexible modulation of the structure and functionality of the NNPs. As illustrated in FIG. 1, PDA may serve multiple concerted functions, for example, as a nanoscale spacer to afford controllable nanogap size, a redox-active coating to promote metal shell growth, a chemical scaffold to exclusively lock molecular probes inside the nanogap for efficient SERS, and a universal adhesive that allows for customized structural integration toward multifunctionality, supported by its unique array of physicochemical properties.

First, PDA deposits from aqueous solution onto virtually any solid substrate, and exhibits strong adhesive property against virtually any solid substrates, forming a conformal coating with precisely controlled thickness in the nanometer scale as a result of self-polymerization of dopamine. Second, the high density of catechol groups imparts reducing activity to PDA, which facilitates in-situ nucleation and deposition of a metallic layer. Third, the spontaneous Michael addition and/or Schiff base reactions of quinone groups in PDA with nucleophilic thiol and amino groups make it possible to encode the nanogaps with molecular probes for SERS and/or metal-enhanced fluorescence

Importantly, the universal adhesion of PDA enables conveniently building up multiple concentric metallic shells (FIG. 1) on diverse inorganic, organic and hybrid functional cores of different sizes, shapes, and chemical compositions, such as Au nanostructures, for example, spherical Au nanoparticles and anisotropic Au nanorods (AuNRs), metal-organic frameworks (MOFs), magnetic polymer nanoparticles (MagNPs), and polymer nanobeads containing magnetic nanoparticles (MagNPs). These may in turn lead to multigap and multifunctional NNPs, which are not readily accessible by conventional wet-chemical synthesis.

The results have demonstrated that the unique set of characteristics of mussel-inspired polydopamine including universal adhesion and diverse chemical reactivity (reducing activity and spontaneous conjugation) enable tailored nanogap engineering of the NNPs in terms of both gap sizes and optical encoding, leading to broadly tunable spectroscopic properties, highly active surface enhanced Raman scattering, and efficient photothermal conversion. Of equal significance is that the polydopamine-based strategy makes it possible for synthesizing well-defined multigap NNPs and multifunctional hybrid NNPs containing chemically different cores (i.e., magnetic nanoparticles and metal-organic frameworks), which are inaccessible by traditional methods and hold great promise for emerging fields such as optoelectronics and theranostics.

In a proof-of-concept study, the inventors have demonstrated that bioconjugated, SERS-encoded magnetoplasmonic NNPs led to efficient magnetic separation, ultrasensitive Raman detection, and effective photothermal killing of a common food-borne pathogen, E. coli. O157:H7.

### EXAMPLE 1

### Materials and Characterization

Dopamine, sodium citrate, potassium gold(III) chloride (KAuCl₄), bicine, hydroxylamine hydrochloride (NH₂OH.HCl), iron(III) chloride hexahydrate (FeCl₃.6H₂O), iron(II) chloride (FeCl₂.4H₂O), ammonium hydroxide, oleic acid, sodium dodecyl sulfate (SDS), styrene, tetradecane, potassium peroxydisulfate (KPS), zirconium(IV) chloride (ZrCl₄), terephthalic acid (H₂BDC), acetic acid, hexadecyltrimethylammonium bromide (CTAB), silver nitrate (AgNO₃), sodium borohydride (NaBH₄), 4-nitrothiophenol (NTP), and bovine serum albumin were purchased from Sigma Aldrich.

Methanol (MeOH) and N,N-Dimethylmethanamide (DMF) were obtained from Fisher Chemical. Hydrogen tetrachloroaurate (III) trihydrate (HAuCl₄.3H₂O) was from Alfa Aesar. Ultrapure water (18.2 MΩ·cm) was purified using a Sartorius AG arium system and used in all experiments. Methoxy-poly(ethylene glycol)-thiol (PEG-SH, 5 kDa) and carboxymethyl-poly(ethylene glycol)-thiol (HOOC-PEG-SH, 3.4 kDa) were purchased from Laysan Bio, Inc. Lissamine rhodamine B ethylenediamine (RhB-NH₂) was purchased from Life Technologies.

LIVE/DEAD® BacLight™ Bacterial Viability Kits was purchased from Thermo Fisher Scientific. The pair of detection/capturing monoclonal antibodies (8B1-C2-B1 and 10C5-H3-B6) was obtained as a gift from Dr. Weihua Lai's group in Nanchang University.

Transmission electron microscopy (TEM) observations were conducted on a Jeol JEM 2010 electron microscope at an acceleration voltage of 300 kV. UV-vis spectra were recorded using a Shimadzu UV1800 spectrophotometer. Fluorescence spectra were collected on a Fluoromax-3 spectrometer (Horiba Scientific). Temperatures of solutions were obtained using FLIR T420 thermal imaging infrared camera. A RENISHAW Raman microscope with WIRE 2.0 software and 632.8 nm (maximum energy: 50 mW) emission line of an air-cooled He—Ne laser was used for SERS measurements. The laser beam with a laser spot size of 2 μm to 5 μm was focused by a 50× objective. A single scan with an integration time of 15 s was performed. The bacterial cells were imaged using laser scanning confocal microscopy (ZEISS LSM 800 with Airyscan). Infrared thermographic images of vesicle dispersions were obtained using FLIR T420 thermal imaging infrared camera.

### EXAMPLE 2

### Synthesis of 20 nm Au Nanoparticles

Au nanoparticles of 20 nm were prepared by citrate reduction of HAuCl₄ in aqueous phase. Typically, a sodium citrate (92 mg) DI-water solution (3 mL) was rapidly injected into a boiling aqueous HAuCl₄ (8 mg in 80 mL of water) solution under vigorous stirring. After boiling for 30 min, the solution was cooled to room temperature.

### EXAMPLE 3

### Synthesis of PDA-Coated 20 nm Au Nanoparticles (Au@PDA)

Typically, as-synthesized 20 nm Au nanoparticles were centrifuged at 7000 rcf (relative centrifugal force) for 15 min. Then, the pellets were redispersed in 2 mL H₂O. A 500 μL sample of the concentrated AuNPs was dispersed in 16 mL of bicine buffer (pH 8.5), followed by adding different amount of dopamine to achieve the corresponding PDA thickness. With concentration of 20 nm Au nanoparticles at 1.8 nM, 0.02 mg/mL, 0.06 mg/mL, and 0.18 mg/mL of dopamine gave rise to 2 nm, 5 nm, and 13 nm thickness of PDA, respectively. Apart from the concentration of dopamine in the solution, the thickness of PDA may also depend on size and number of cores onto which the PDA is to be deposited on. Generally, at the same dopamine concentration, larger size of cores or larger number of cores results in thinner PDA coating. The reaction solution was stirred for 8 h. The purple product was purified by centrifugation and was stored in 2 mL H₂O at 4° C. for further use.

### EXAMPLE 4

### Synthesis of 50 nm Au Nanoparticles

50 nm AuNPs were prepared using a seeded-growth method. Briefly, 50 mL water was added into a 100 mL round-bottom flask. 2 mL of seed AuNP solution containing Au nanoparticles prepared from Example 2 and 200 μL of 0.2 M NH₂OH.HCl were added into this flask consecutively. Afterwards, 3 mL of 0.1 wt% HAuCl₄ was added dropwise into the solution under vigorous stirring followed by 30 min reaction at room temperature. A gradual color change from light red to dark red was observed. Finally, concentration of the sodium citrate was adjusted to 1 mM. After reacting for another 2 h, nanoparticle dispersion was stored at 4° C. for further use.

### EXAMPLE 5

### Synthesis of PDA-coated 50 nm Au Nanoparticles (Au(50 nm)@PDA)

Typically, 50 nm Au nanoparticles were centrifuged at 1200 rcf for 15 min. Then, the pellets were redispersed in 1 mL H₂O. The concentrated AuNPs was dispersed in 16 mL of bicine buffer (pH 8.5), followed by adding dopamine to achieve required PDA thickness. The reaction solution was stirred for 8 h, and the purple product was purified by centrifugation.

### EXAMPLE 6

### Synthesis of Au Nanogapped Nanoparticles (Au NNPs)

Typically, 80 μL of Au@PDA (0.6 nM) was added into 2 mL H₂O at 50° C. After stirring for 2 min, 100 μL of 2.5 mM KAuCl₄ was injected, followed by 50 μL of 0.2 M NH₂OH.HCl. The color of the solution changed from light red to dark purple immediately. The reaction solution was stirred for 2 min. After cooling down, 50 μL of PEG-SH (5 kDa, 10 mg/mL) was added into the solution to further stabilize the nanogapped nanoparticles. Finally, the product was purified by centrifugation. For nanogapped nanoparticles of different PDA thickness, the amount of Au precursor was changed accordingly.

### EXAMPLE 7

### Synthesis of Multi-Shell Au Nanostructure

For double-shell NNPs, the single-shell NNPs were used as a core and the procedures as described above may be repeated for the growth of the Au shell. Typically, Au(50nm)@Single Shell was dispersed in 4 mL of bicine buffer (pH 8.5), followed by adding dopamine (0.1 mg/mL). The reaction solution was stirred for 8 h and the resultant Au(50nm)@Single Shell@PDA was purified by centrifugation. Next, the obtained product was added into 2 mL of H₂O at 50° C. After stirring for 2 min, 120 μL of 2.5 mM KAuCl₄ was injected, followed by 60 μL of 0.2 M NH₂OH.HCl. The reaction solution was stirred for 2 min and 50 μL of PEG-SH (10 mg/mL) was added into the solution to further stabilize the double-shell NNPs. Finally, the product (Au(50nm)@Double Shells) was purified by centrifugation. In the synthesis of triple-shell NNPs, the double-shell NNPs were used as the cores.

### EXAMPLE 8

### Synthesis of Raman Dye Labelled Au NNPs (Au@PDA@Au)

Typically, Au@PDA nanoparticles were dispersed in 2 mL of bicine buffer (pH 8.5) under continuous stirring, followed by adding 0.5 mg/mL RhB-NH₂ solution. After reacting for 24 h, Au@PDA-RhB nanoparticles were collected by centrifuge and washed with DI water for three times. The number of conjugated dyes was determined by the fluorescence intensity of unbound RhB molecules in the supernatant and can be controlled by the feeding ratio of RhB and Au@PDA. For example, in case of Au@PDA-2, a conjugation efficiency of 68% was achieved when the feeding ratio was 300:1. These Raman dye labelled Au@PDA-RhB nanoparticles were used as the cores to construct Au NNPs (Au@PDA-RhB@Au) for SERS detection.

### EXAMPLE 9

### Calculation of Enhancement Factor (EF)

The EF of individual NNPs was determined by computing the ratio of SERS to normal Raman scattering of RhB using the following equation, EF=(I_{SERS}×C_{Normal})/(I_{Normal}×C_{SERS}), where I_{SERS} and I_{Normal} are the Raman intensities at 1647 cm⁻¹ for nanogapped Au nanoparticles and pure RhB solution, C_{SERS} and C_{Normal} the concentrations of RhB on NNPs and in pure solution. C_{SERS} was calculated using the equation C_{SERS}=N×C_{Au}, where N is the number of RhB in the NNPs.

### EXAMPLE 10

### Synthesis of Au Nanorods (AuNR)

A seed-mediated method was used to prepare the Au nanorods. Typically, two steps were included. First, gold seeds were synthesized as reported previously. An HAuCl₄ solution (250 μL of 10 mM) was added to the cetyltrimethylammonium bromide (CTAB) solution (9.75 mL, 0.1 M); then, under vigorous stirring, a freshly prepared NaBH₄ solution (0.6 mL, 0.01 M) was injected. The solution color changed immediately from yellow to dark brown. After stirring for 5 min, the mixture solution, as seed solution, was kept for at least 1 h at room temperature before it was used in the next step. Second, Au nanorods were synthesized in a growth solution. An HAuCl₄ solution (500 μL of 10 mM) was added to 9.5 mL of the CTAB solution. The mixture solution was incubated at 40° C. for 10 min. Then AgNO₃ solution (0.1 M), dopamine hydrochloride solution (0.2 M), and seed solution were added sequentially. The resulting growth solution was mixed thoroughly and kept undisturbed in a water bath set at 40° C. for 3 h.

### EXAMPLE 11

### Synthesis of PDA-Coated Au Nanorods (AuNR@PDA)

Typically, Au nanorods were centrifuged at 8500 rcf for 15 min. Then, the pellets were redispersed in 1 mL H₂O. The concentrated AuNRs was dispersed in 16 mL of bicine buffer (pH 8.5), followed by adding dopamine to achieve required PDA thickness. The reaction solution was stirred for 8 h and the dark brown product was purified by centrifugation.

### EXAMPLE 12

### Synthesis of UiO-66 Nanoparticles

Nanosized UiO-66 particles were prepared by dissolving 4 mM ZrCl₄ and 4 mM H₂BDC in a mixture of dimethylformamide (DMF) and EtOH containing acetic acid. The reaction vial was capped and placed into an oven preheated at 100° C. for 12 h. The product was collected by centrifugation and then washed three times with DMF and MeOH, respectively. The product was suspended in MeOH.

### EXAMPLE 13

### Synthesis of Plasmonic Nanogapped UiO-66 Nanoparticles

Typically, 10 mL of UiO-66 nanoparticles was dispersed in 30 mL of bicine buffer (pH 8.5), followed by adding 10 mg of dopamine. The reaction solution was kept stirring for 12 h. The light brown product (UiO-66@PDA) was purified by centrifugation. Then, a proper amount of UiO-66@PDA was added into 10 mL H₂O at 50° C. After stirring for 2 min, 1.2 mL of 2.5 mM KAuCl₄ was injected, followed by 120 μL of 0.2 M NH₂OH.HCl. The color of the solution changed from light brown to bluish green immediately. The reaction solution was stirred for 2 min and 50 μL of PEG-SH (10 mg/mL) was added into the solution to further stabilize the nanogapped nanoparticles. Finally, the product (UiO-66@PDA@Au) was purified by centrifugation. This procedure is repeated one more time to achieve plasmonic gapped nanoparticles (UiO-66@PDA@Au@PDA@Au).

### EXAMPLE 14

### Synthesis of MagNPs

Polystyrene-trapped magnetic iron oxide nanoparticles (MagNPs) were prepared by emulsion polymerization. FeCl₃.6H₂O (2.4 g) and FeCl₂.4H₂O (0.982 g) were dissolved in 10 mL DI water under N₂ gas with vigorous stirring at 80° C. Then, 5 mL of ammonium hydroxide was added rapidly into the solution. The color of solution turned to black immediately. After 30 min, 3 mL of oleic acid was added and the suspension was kept at 80° C. for 1.5 h. The obtained magnetite nanoparticles were washed with water and MeOH until pH became neutral.

Magnetite nanoparticles (0.5 g) obtained were added into 12 mL water containing 10 mg sodium dodecyl sulfate (SDS), and the mixture in ice-water bath was treated with ultrasound for 10 min to obtain miniemulsion of magnetite nanoparticles. Meanwhile, a styrene emulsion was prepared using 5 mL styrene, 50 mg SDS, 40 mL water, and 0.033 mL tetradecane.

Miniemulsion of magnetite nanoparticles and 5 mg potassium persulfate (KPS) were added to a three-neck flask and stirred for 30 min at 500-600 rpm in N₂ atmosphere. Afterwards, 10 mL of styrene emulsion was added into the mixture, and the flask was placed in 80° C. water bath and maintained for 20 h to obtain MagNPs.

This as-fabricated MagNPs was collected with a magnet and redispersed in H₂O, and the collection-redispersion cycle was repeated three times before dispersing the MagNPs in 10 mL H₂O for further usage.

### EXAMPLE 15

### Synthesis of Magnetic NNPs

Briefly, 50 μL of MagNP was dispersed in 16 mL of bicine buffer (pH 8.5), followed by adding 10 mg of dopamine to achieve the required PDA thickness. The reaction solution was kept stirring for 8 h. The dark brown product (MagNP@PDA) was purified by centrifugation and stored in 1 mL of H₂O. To fabricate the first Au shell, 100 μL of MagNP@PDA was added into 10 mL H₂O at 50° C.

After stirring for 2 min, 1.2 mL of 2.5 mM KAuCl₄ was injected, followed by 1.2 mL of 0.2 M NH₂OH.HCl.

The obtained product (MagNP@PDA@Au) was collected and further dispersed in bicine buffer to undergo another cycle of PDA coating and metallization. Eventually, the color of the solution changed from brown to green. The resulting magnetic NNPs were surface modified with bifunctional HOOC-PEG-SH (3.4 kDa).

### EXAMPLE 16

### Surface Modification of Magnetic NNPs

The magnetic NNPs were collected by centrifuge and dispersed in 5 mL of 2-(Nmorpholino)ethanesulfonic acid (MES) buffer (pH 5.5). To activate the carboxylic acid group on the surface of these particles, 0.2 mL of 1-ethyl-3-(3-(dimethylamino)propyl)carbodiimide (EDC, 5 mg/mL) and sulfo-N-hydroxysuccinimide (NHS, 5 mg/mL) were added to the solution and incubated for 30 min.

The excess EDC and NHS were removed by centrifuge. Then, the detection monoclonal antibody (8B1-C2-B1) (30 μg/mL) in borate saline buffer (pH 8.0) was quickly added to the activated particles with gentle stirring for 3 h at room temperature. Finally, 1 mL of bovine serum albumin (BSA) solution (2 mg/mL) was added to the mixture to block the unreacted sites for 1 h. The free reactants were removed by centrifuge. The bioconjugated magnetic NNPs were stored at 4° C. before use.

### EXAMPLE 17

### Bacterial Culture

E. coli O157:H7 (ATCC 43888) and other bacteria were cultured in Luria-Bertani medium for 20 h at 37° C. before use. The number of viable cells was determined by plate count. The cells were treated with 0.3% formaldehyde for 24 h to kill all bacteria. The inactivated bacteria were collected by centrifugation at 4000 rpm and resuspended in 0.01 M phosphate-buffered saline (PBS) (pH 7.4). Finally, these bacteria were serially diluted to the desired concentrations with 0.01 M PBS (pH 7.4) for further use.

### EXAMPLE 18

### Detection and Photothermal Killing of E. coli O157:H7 Using Magnetic NNPs

A 25 μL amount of antibody-conjugated magnetic NNPs (0.5 mg/mL) was added to 1 mL samples containing 10, 10², 10³, 10⁴, 10⁵, 10⁶, 10⁷, and 10⁸ CFU/mL of E. coli O157:H7 or other bacteria. The mixture was gently shaken for 30 min and placed in a magnetic field for 10 min to separate the immune complex of E. coli O157:H7 and magnetic NNPs. The complex in 50 μL of PBS was added to the capture antibody (10C5-H3-B6)-immobilized 96-well microtiter plate and incubated for 30 min at room temperature. The plate was then washed three times with 0.01 M PBS (pH 7.4) containing 0.05% Tween 20. Then the plate was placed under Raman microscopy for spectral collection in the range of 800 to 1800 cm⁻¹ using 50 mW of laser power. The calibration curve was plotted using the peak intensities of NTP at 1341 cm⁻¹ vs the concentration of E. coli O157:H7 (10 to 10⁸ CFU/mL). The photothermal treatment of the captured bacteria was conducted by exposure to an 808 nm laser (1 W/cm²) for 15 min. The temperature was monitored by an infrared camera. The bacteria were stained by LIVE/DEAD BacLight bacterial viability kits in the dark for 15 min and then imaged using laser scanning confocal microscopy.

### EXAMPLE 19

### NIR Laser-Induced Photothermal Conversion

The aqueous solution of nanogapped Au nanoparticles with same particle concentration (1 nM) or optical density (ODs) (1.0) were irradiated by a 808 nm laser at a power density of 1 W/cm² for 5 min. The laser spot was adjusted to cover the whole surface of the samples. The temperature elevation of the aqueous solutions was recorded as a function of the amount of time they were exposed to laser irradiation. Temperature and thermographic images were taken by a FUR thermal camera at 30 s intervals.

### EXAMPLE 20

### Results and Discussion

Dopamine undergoes consecutive oxidation, intramolecular cyclization, and oligomerization/self-assembly in alkaline conditions, leading to highly crosslinked adhesive PDA that is able to form a conformal layer of coating on colloidal particles of diverse surface composition.

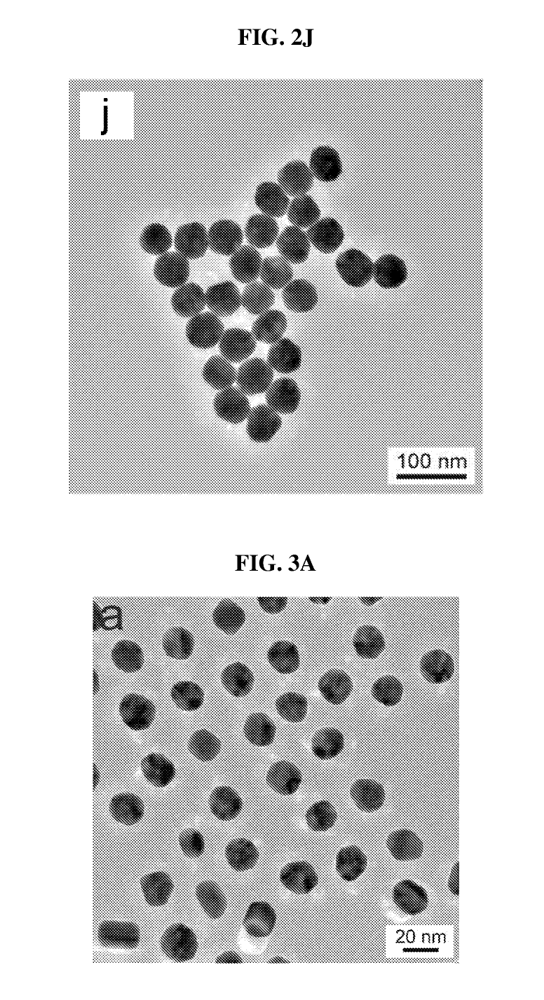

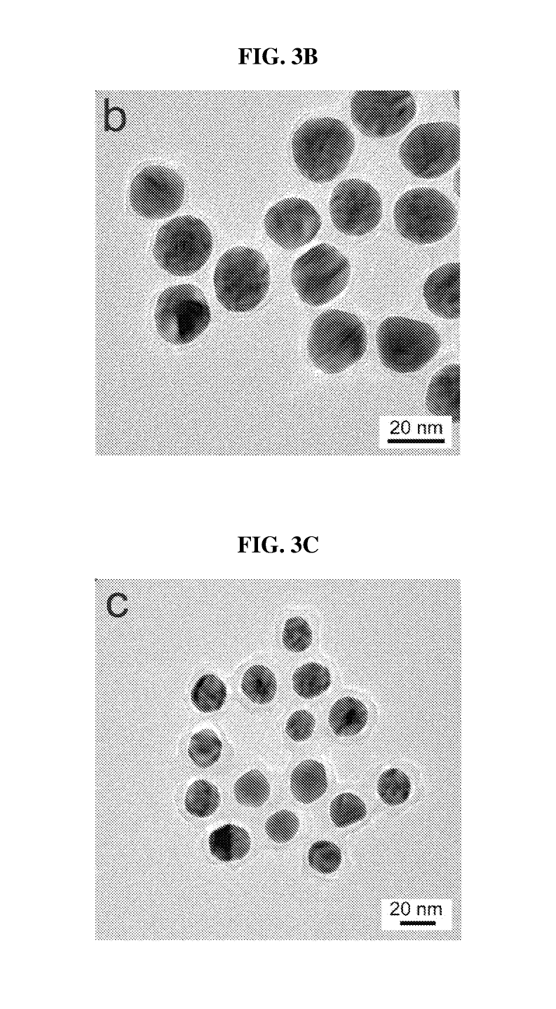

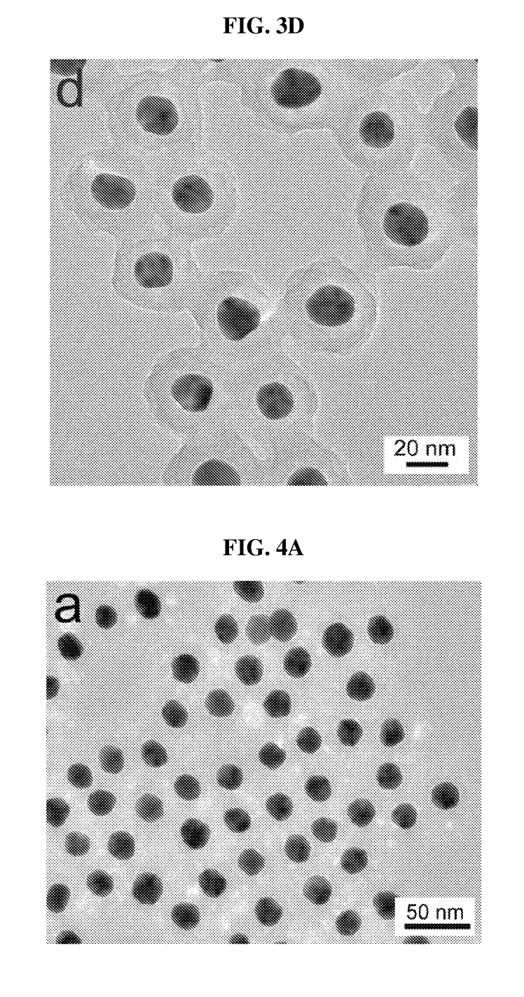

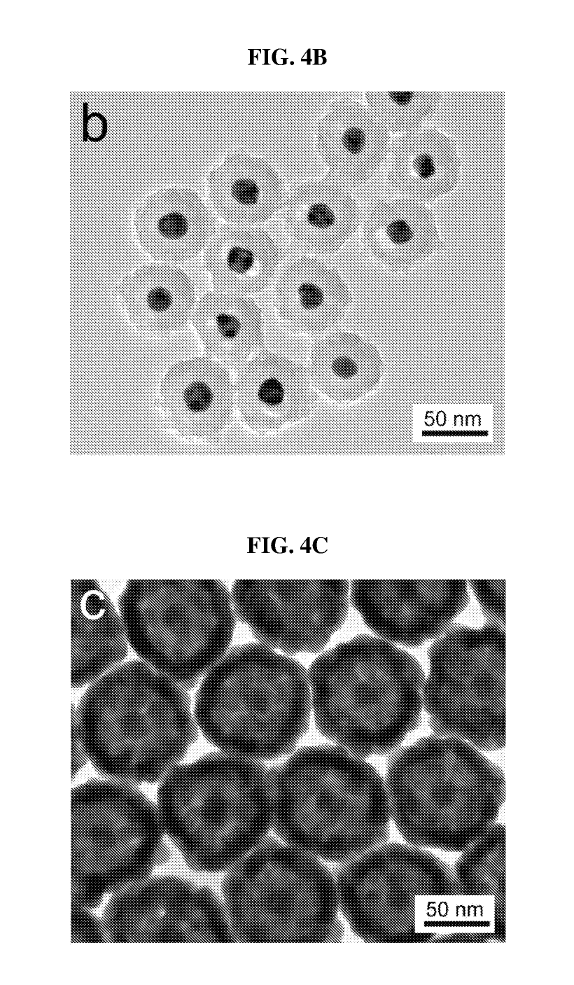

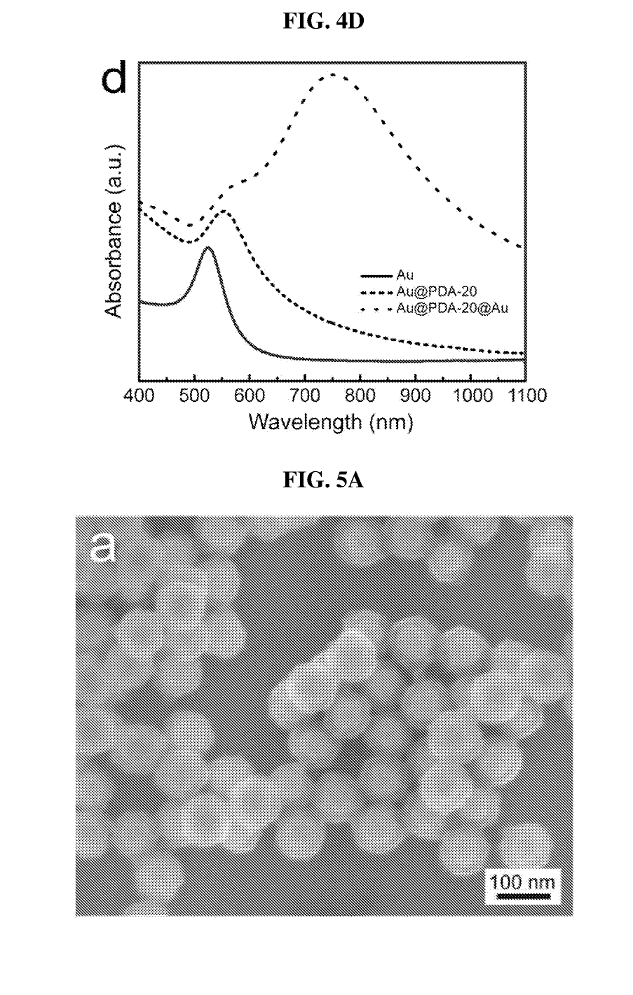

The inventors have found that the deposition of PDA coating on citrate-stabilized Au nanoparticles can be controlled by the starting concentration of dopamine. Transmission electron microscopy (TEM) images (FIG. 2A and FIG. 3A to 3D) clearly reveal that monodisperse PDA coated Au nanoparticles (Au@PDA) with a PDA thickness of 2 to 13 nm were produced after a 8 h reaction in bicine buffer (pH 8.5), with a dopamine concentration from 0.025 to 0.2 mg/mL. Note that, although the PDA thickness in one cycle of reaction culminates up to 13 nm, it can be further increased to tens of nanometers by applying multiple coating cycles (FIG. 4A to 4D).

PDA carries a high density of catechol groups, which can induce localized reduction of metal precursors. The results obtained herein have shown that successive addition of KAuCl₄ and NH₂OH in presence of Au@PDA at 50° C. gave rise to well-defined Au NNPs (FIG. 2D to 2F). A key finding here is that the nanogap size of the NNPs matches the thickness of PDA coating in Au@PDA, which, together with the flexibly tunable PDA thickness, makes it possible to systematically tailor the nanogap size in a broad range. Apparently, complexation and reduction of AuCl₄ ions by catechol groups facilitates in-situ nucleation, which confines the subsequent growth of a Au shell on the surface of the PDA layer.

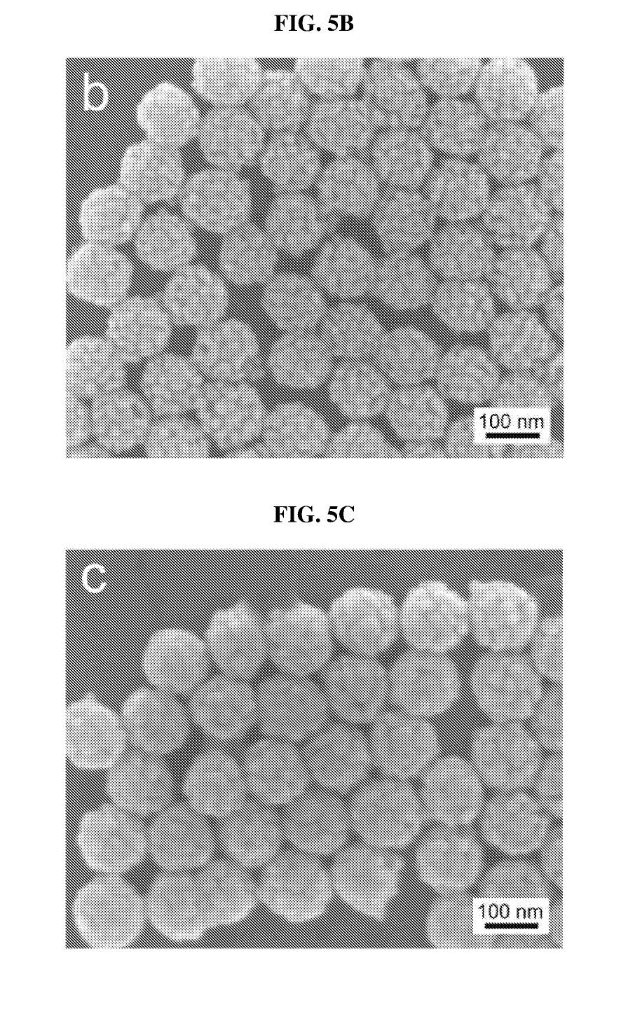

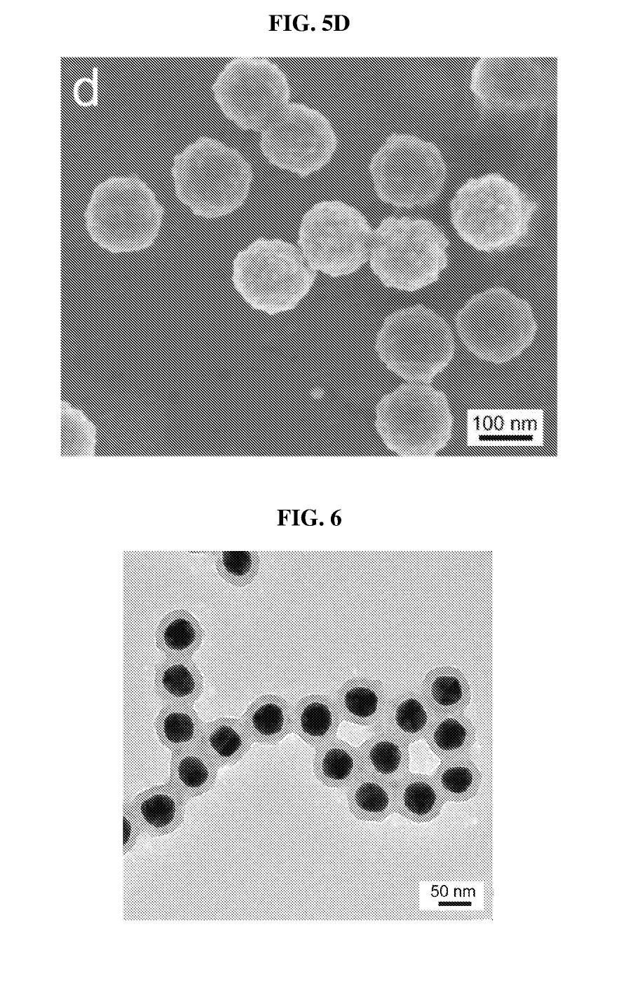

This analysis is supported by the rapid completion of colorimetric changes within 1 min during the reaction, which is a result of LSPR shifts as discussed herein. Scanning electron microscopy (SEM) observation (FIG. 5A to 5D) reveals that the continuous growth of isolated domains during this process resulted in a complete shell when an increasing amount of KAuCl₄ precursor was introduced.

This is in contrast to use of HAuCl₄, which may cause degradation of PDA instead of experiencing localized reduction. The inventors reason that use of HAuCl₄ rather than KAuCl₄ induced an acidic environment, in which catechol groups have weak reducing power. Importantly, the universal adhesion of PDA makes this PDA-based strategy compatible with nanoparticles of different sizes, shapes and surface chemistry.

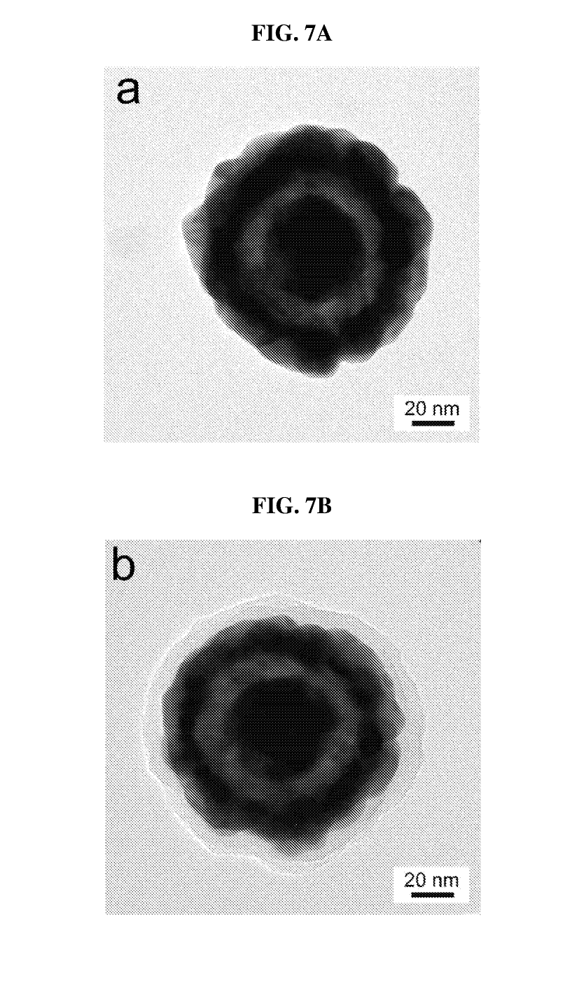

More interestingly, the strategy presented herein affords access to multigap NNPs consisting of multiple concentric nanoshells surrounding the core. NNPs are first prepared on 50 nm Au nanoparticles with a 13 nm nanogap (FIG. 2G and FIG. 6). By repeating the cycles of PDA coating and metallization, NNPs with two or three plasmonic shells were obtained, as shown in FIGS. 2H, 2I, and FIGS. 7A and 7B. Such multigap NNPs were theoretically predicted to function as a series of optical condensers to direct light toward the center of their structures, inducing a dramatically amplified local field in the gap between the nanoparticle core and its adjacent shell. The inventors' PDA-based approach therefore provides interesting opportunities for in-depth experimental studies by offering flexible control over the structure of multigap NNPs in terms of gap size and the number of shells.

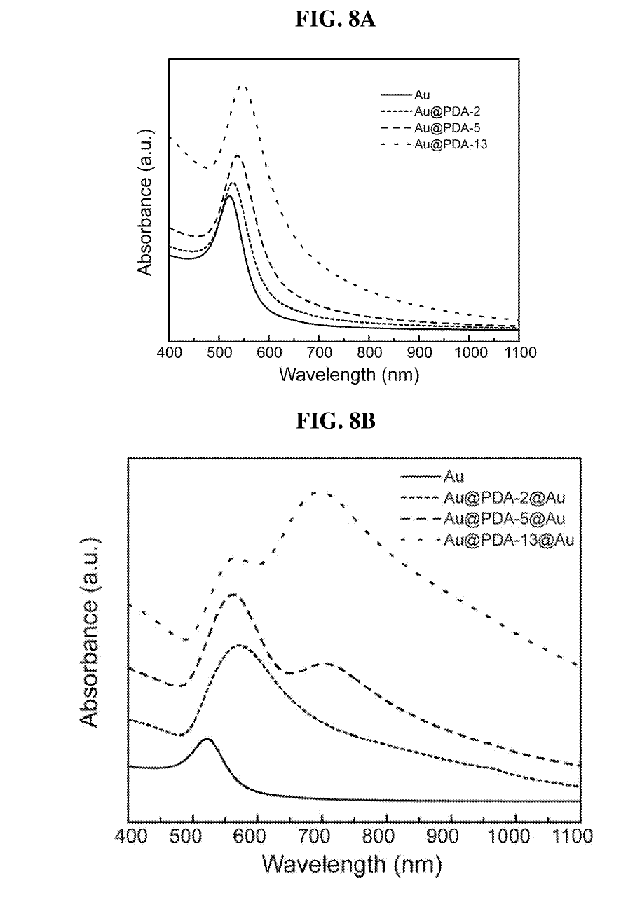

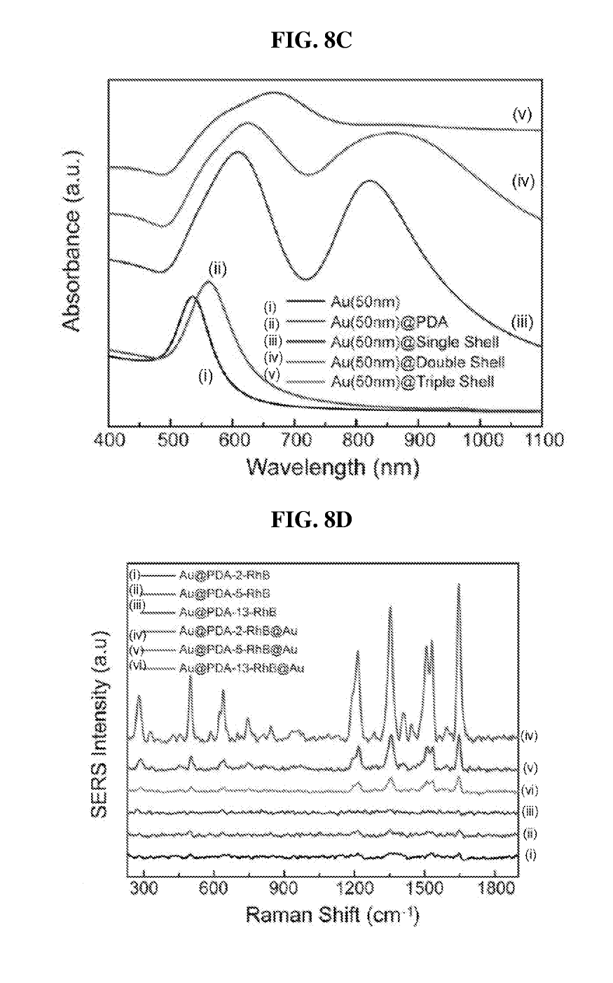

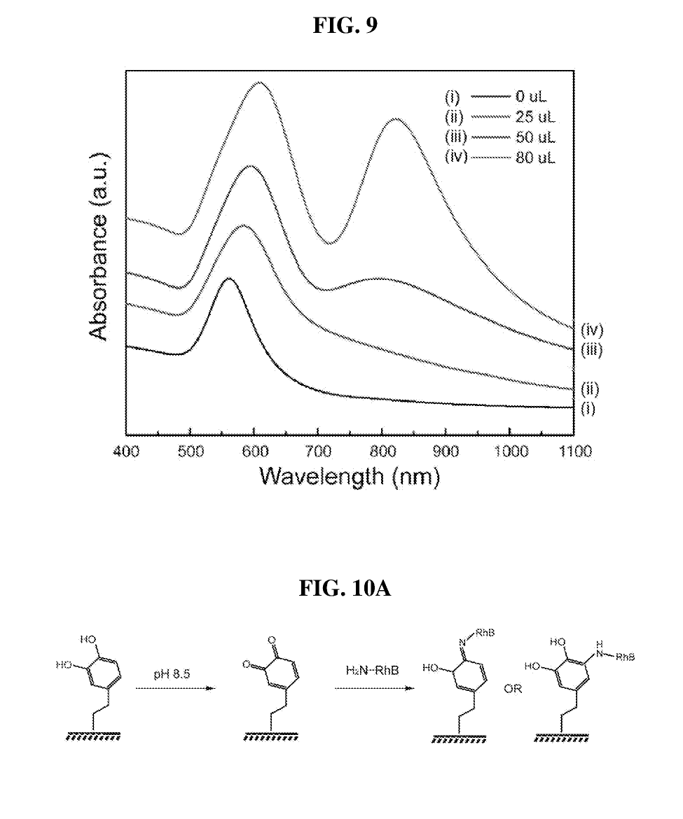

The LSPR of plasmonic nanostructures is highly sensitive to changes in structural parameters and local dielectric environment. Au nanoparticles of 20 nm with an original LSPR centered at 522 nm experienced a gradual red-shift to 530, 538, and 548 nm (FIG. 8A) for PDA coating thicknesses of 2, 5, and 13 nm, respectively, due to the larger refractive index of PDA in comparison with that of water. Au NNPs with a 2 nm gap showed a further spectral shift to 575 nm (FIG. 8B) because of the strong coupling of closely arranged core and shell. When the gap expanded to 5 and 13 nm, a new resonance peak around 750 nm appeared and became dominant in the NNPs with a 13 nm gap due to a greater extinction coefficient of the larger Au shell. Similarly, in the case of the NNPs structured with a 50 nm core, 13 nm gap, and 15 nm shell, hybridization of the core and shell plasmon modes gave rise to two separate peaks at 610 and 823 nm (FIG. 8C), which further red-shifted for the double-shell NNPs and eventually leveled off in the triple-shell NNPs to cover almost the entire visible and NIR spectral range. In line with the SEM observation (FIG. 5A to 5D), when the Au shell gradually closes up during the growth, a weak shoulder at 823 nm (FIG. 9) emerges and evolves to a distinct strong peak. The confined electromagnetic field surrounding the plasmonic nanostructures plays a leading role in SERS of the molecules in their close proximity. Strongly coupled plasmonic nanostructures separated by a nanogap junction of less than 5 nm lead to a dramatically amplified local field in the nanogap, representing an efficient SERS hot-spot.

While a number of chemical and self-assembly methods have been proposed to generate nanogaps, most of the previous methods lack the ability to precisely position molecular probes inside the hot-spots, instead relying on random diffusion of the probes, which becomes a major challenge for using SERS nanoprobes in quantitative detection. In the design disclosed herein, spontaneous covalent coupling of nucleophilic thiol and amine groups with quinone groups in PDA (FIG. 10A and 10B) provides an opportunity for stable, quantitative molecular fixation inside the SERS-active nanogap. Rhodamine B (RhB) carrying a primary amine group was selected as a model Raman probe to be tagged on the PDA layer prior to the deposition of the Au nanoshell. The number of RhB molecules anchored can be controlled by the feeding ratio of RhB and the nanoparticle core.

FIG. 8D shows the Raman spectra of NNPs and control samples containing an average of about 200 RhB molecules under the excitation of a 633 nm laser. The excellent SERS activity of the NNPs with a 2 nm nanogap was confirmed by an enhancement factor of 8.8×10⁷, which dropped to 2.0×10⁷ and 9.6×10⁶ for the 5 and 13 nm gap, respectively. Incubating of the as-prepared SERS-encoded NNPs in aqueous medium did not lead to any obvious change of Raman intensity over time, indicating that the covalent linkage and the complete Au shell locked the RhB tags inside the nanogap.

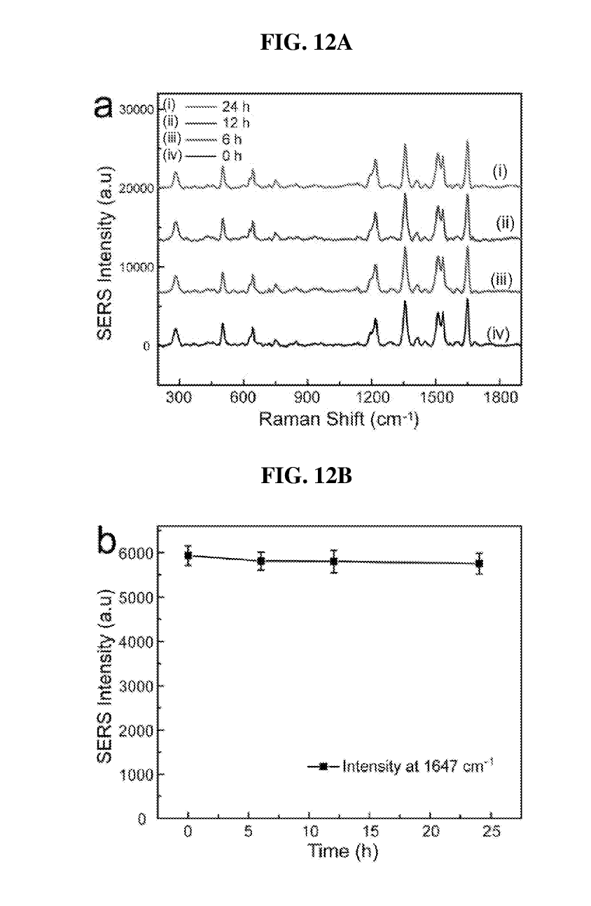

Also important is that Raman intensity shows linear dependence on the number of RhB molecules attached (FIG. 11), offering the possibility of tailoring the Raman signal quantitatively. Moreover, Raman intensity of the as-prepared probes remains constant in aqueous medium (FIG. 12A and 12B), suggesting that the Raman tags are locked inside the nanogap by the covalent linkage and the complete Au shell. In contrast, the NNPs without the RhB probe loaded in the nanogap showed only a featureless background spectrum (FIG. 24) at the same condition. Control nanoparticles, i.e., the RhB-tagged Au@PDA nanoparticles without the nanoshell, also exhibit negligible signals (FIG. 8D), highlighting the significance of the nanogap hot-spots.

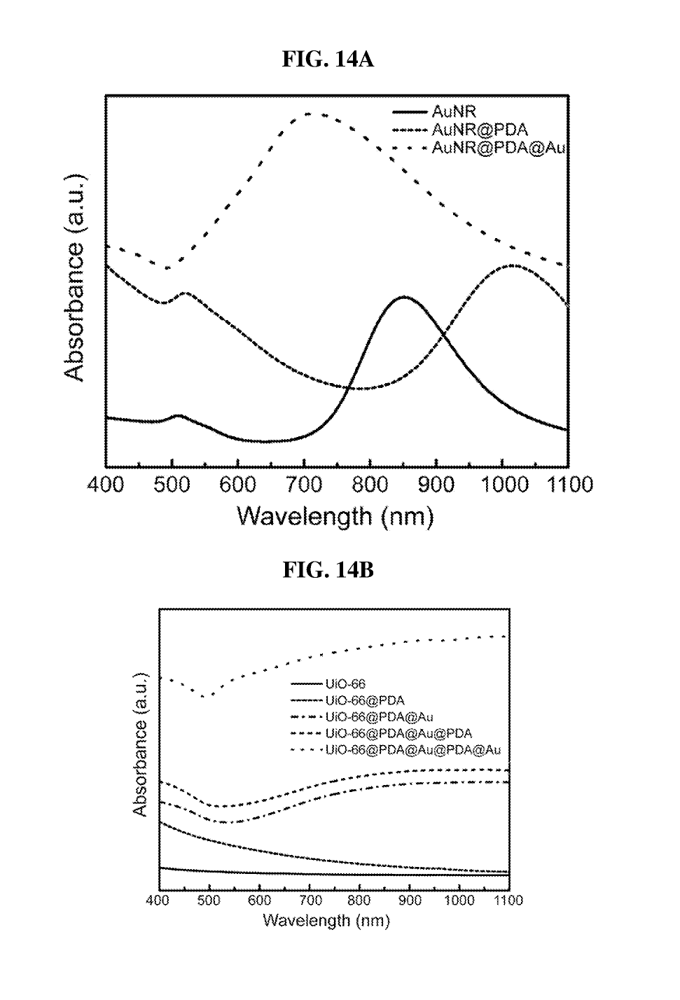

The universal adhesion of PDA offers the possibility of growing Au shells on nanoparticles of different sizes, shapes, and compositions. FIG. 13A to 13F show the NNPs templated by AuNRs and MOF nanocrystals. The rigid and conformal nature of the PDA coating gives rise to anisotropic NNPs that retain the shape of the nanocrystal cores. When elongated AuNRs are used as the core, ellipsoidal NNPs can be easily produced (FIG. 13A to 13C, FIGS. 14A and 14B).

The inventors also investigated the synthesis of hybrid analogues of Au NNPs with nonmetallic cores, i.e., MOF nanocrystals with well-defined shapes. When octahedral UiO-66 nanocrystals (FIG. 13D), formed by 1,4-benzenedicarboxylic acid (H₂BDC) as organic linkers and zirconium(IV) as metal nodes, are chosen as the MOF core, an integral octahedral Au shell can be readily formed on the MOF core (FIG. 13E). Furthermore, a plasmonic nanogap between two adjacent metallic nanoshells (FIG. 13F, FIGS. 14A and 14B) is generated after another cycle of PDA coating and metallization, demonstrating the flexibility of the present strategy in nanogap engineering and structural integration.

The compatibility of a PDA coating with diverse core materials further encouraged the inventors to develop multifunctional NNPs with a magnetic core and a double-shell plasmonic nanogap. The uses of magnetic nanomaterials in bioseparation and bioimaging are representative examples of translation bionanotechnology. Imparting magnetic properties to NNPs leads to magnetoplasmonic nanostructures of considerable interest for biosensing, theranostic, and catalytic applications.

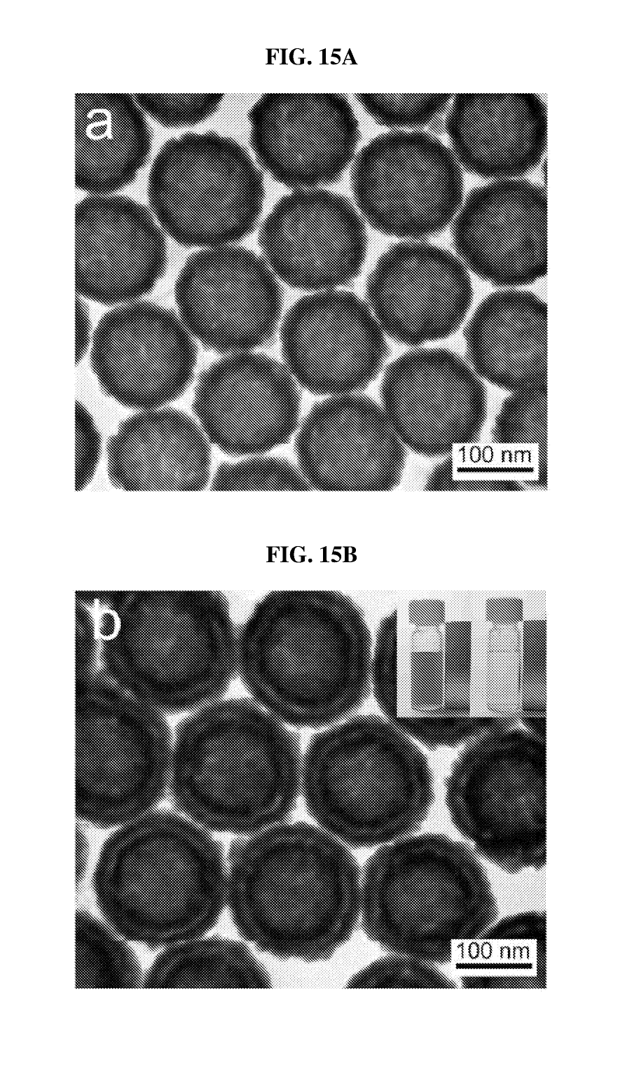

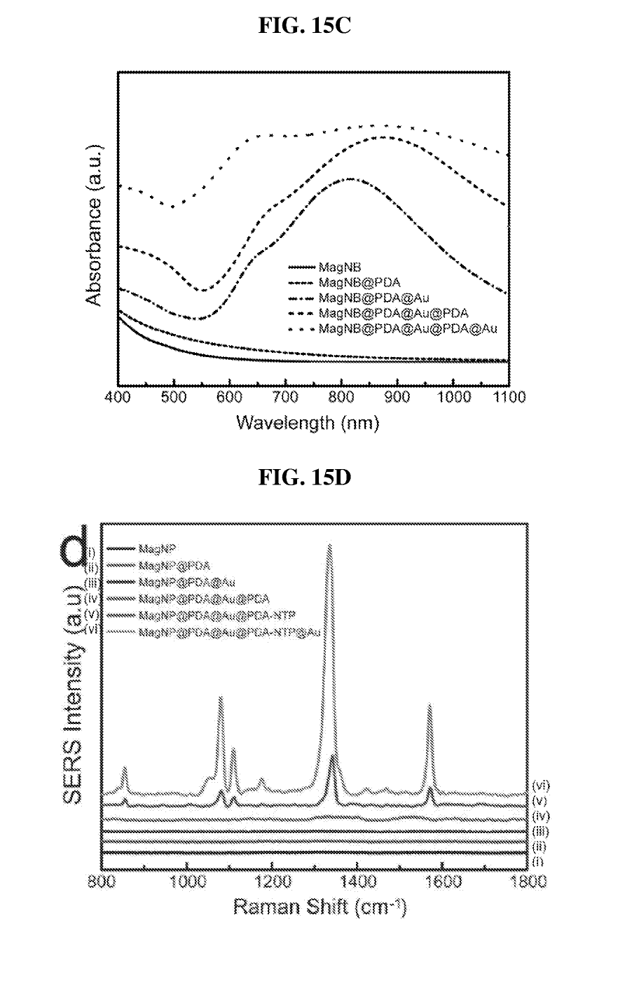

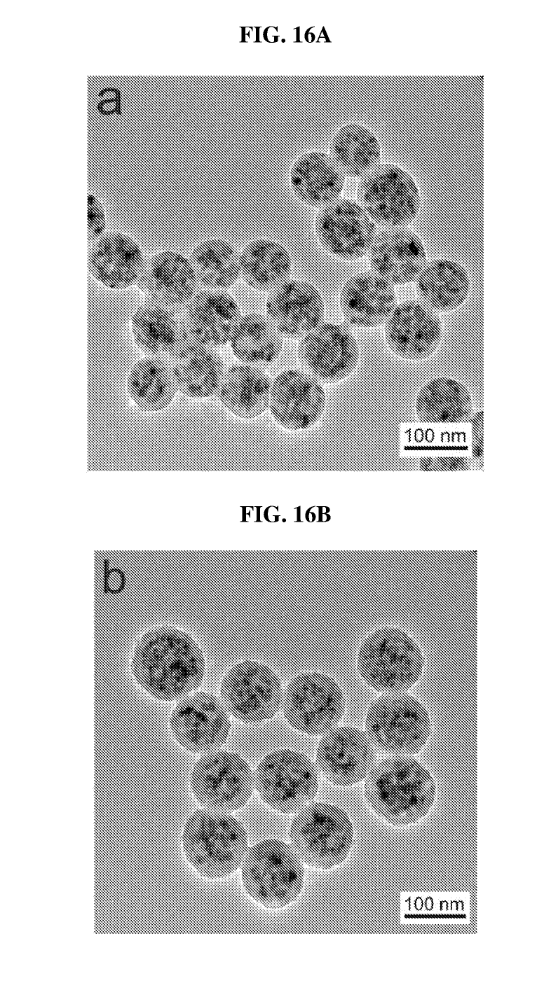

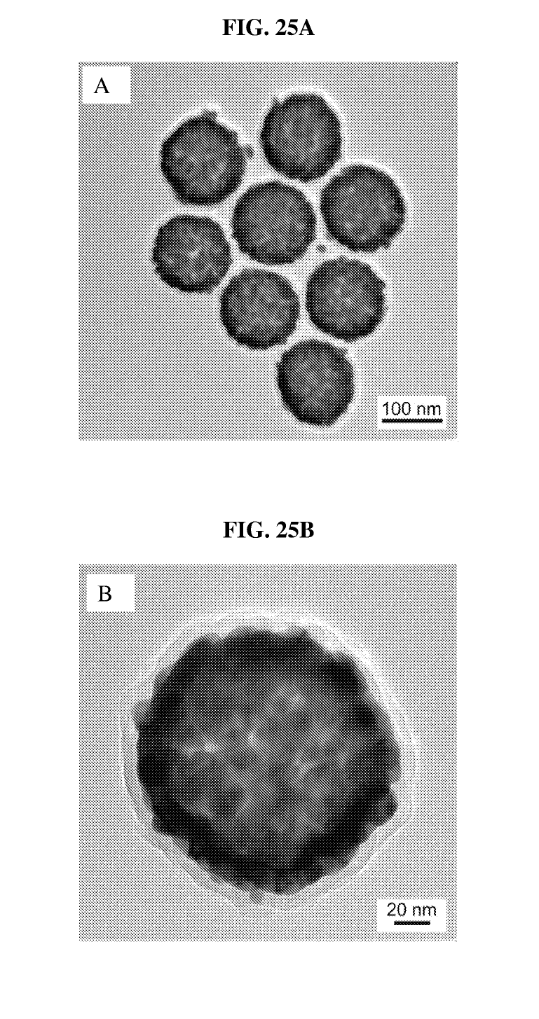

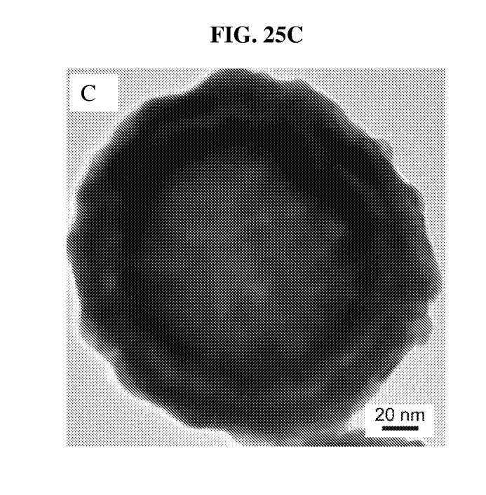

TEM images in FIG. 15A to 15D, FIG. 16A 16B, and FIG. 25A to 25C confirm that the synthesis protocol disclosed herein is also applicable for magnetic polystyrene nanoparticles. The growth of two consecutive layers of 15 nm Au nanoshells led to a uniform nanogap around the magnetic core (FIGS. 15A and 15B). The resulting NNPs retained the magnetic response of the core and can be easily collected by an external magnet, as shown in the inset of FIG. 15B. MagNPs and PDA-coated MagNPs show a broad absorbance in the visible-NIR region (FIG. 15C). When the first Au shell was built, a strong LSPR peak appeared at 834 nm. A PDA coating on the Au shell resulted in a red-shift to 868 nm. After the second Au shell was grown, the LSPR became even broader to cover the entire visible and NIR spectral range between 400 and 1100 nm (FIG. 15C). Introducing a Raman tag, i.e., 4-nitrothiophenol (NTP), in the sub-5 nm nanogap gave rise to Raman signals 5.4-fold stronger than that of the MagNP@PDA@Au with only one Au shell (FIG. 15D), which also led to strong signal amplification because NTP is able to anchor on the Au nanoshell via the Au—S bond. Note that locking Raman tags inside the nanogap insulates them from interfering factors from the surrounding environment, which is essential for quantitative detection.

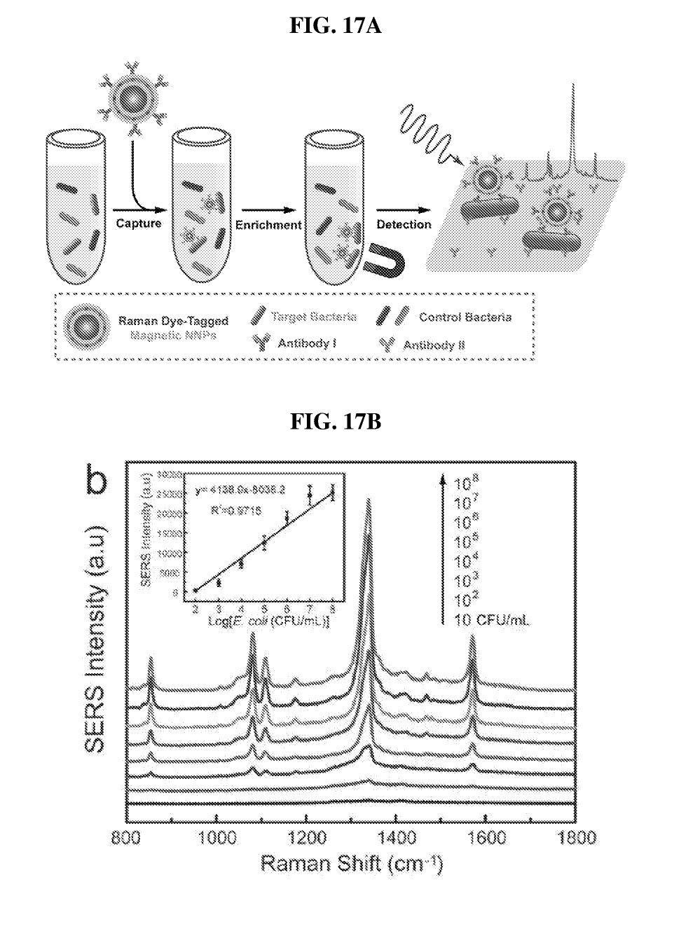

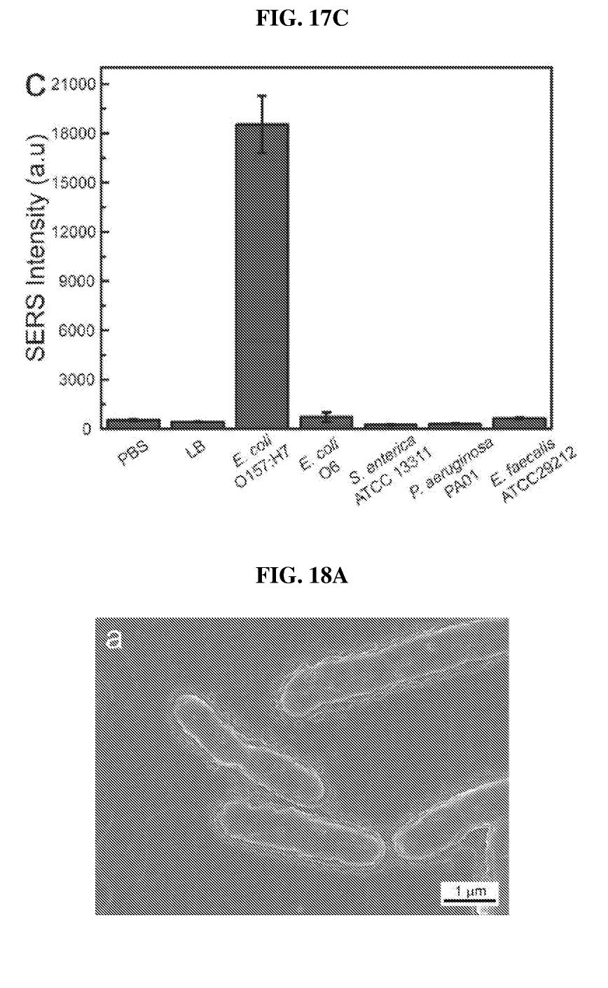

SERS-encoded magnetoplasmonic NNPs offer the possibility of combining magnetic separation, Raman spectroscopy for ultrasensitive detection, and photothermal transduction. As a proof of concept, the inventors applied the NNPs for the quantitative immunoassay of a common food-borne bacterial pathogen, i.e., E. coli O157:H7 (FIG. 17A to 17C). Two antibodies specific to E. coli O157:H7 were attached to PEGylated NNPs and a 96-well microtiter plate blocked by bovine serum albumin, respectively.

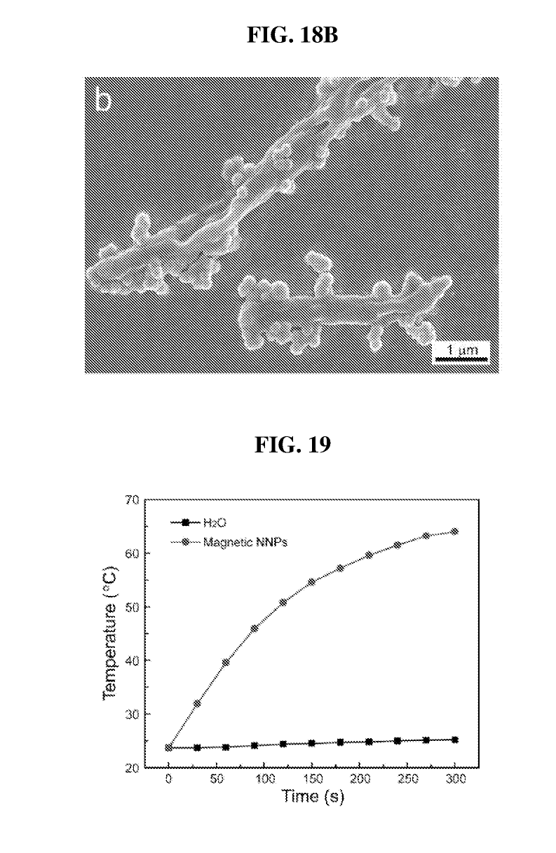

In the assay (FIG. 17A), antibody-conjugated magnetic NNPs were first introduced in the samples containing spiked E. coli O157:H7, which were magnetically captured and enriched afterward upon the binding of the NNPs on the bacteria (FIGS. 18A and 18B). The labeled bacteria were then exposed to the antibody-coated substrates. Finally, the substrates with immobilized bacteria were subjected to Raman detection of the SERS-encoded NNP probes. The results obtained (FIG. 17B) show that the SERS intensity gradually increases in a bacterial concentration range of 10 to 10⁸ CFU/mL with a high sensitivity (about 10² CFU/mL). As summarized in FIG. 17C, both buffer controls (PBS and Luria-Bertani culture medium) and other bacteria such as E. coli O6, S. enterica ATCC 13311, P. aeruginosa PA01, and E. faecalis ATCC 29212 of the same concentration gave rise to negligible signals. Apparently, the high specificity of the antibodies and surface blocking strategies gave rise to excellent selectivity in the immune sandwich assay.

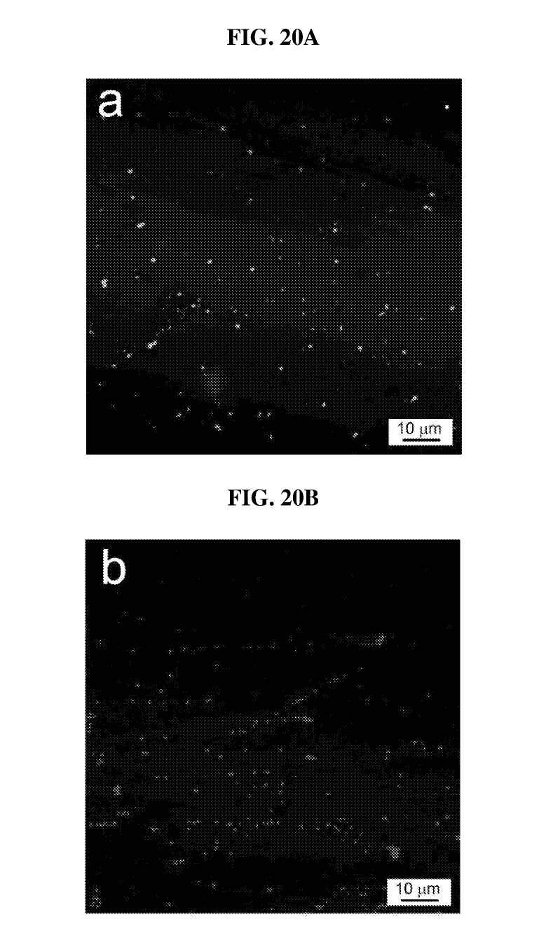

The magnetoplasmonic NNPs are also highly efficient photothermal transducers that lead to a rapid temperature increase of 39.5° C. upon 5 min of laser irradiation, as shown in FIG. 19. Live/dead analysis with the BacLight kit utilizing a mixture of SYTO 9 and propidium iodide (PI) for fluorescence staining shows that only dead bacteria labeled with red PI dye are observed after laser irradiation of 15 min (FIGS. 20A and 20B), confirming the nearly 100% bacterial killing by the photothermal effect of the magnetic NNPs. In comparison with platforms reported previously, magnetoplasmonic NNPs in this work not only allow for highly sensitive detection of pathogens by collective magnetic enrichment and excellent SERS activity but also lead to effective killing of the separated pathogen by the photothermal effect of the NNPs. The easy structural integration of diverse functional cores in the NNPs makes it possible for developing multifunctional plasmonic nanostructures, which are of particular interest for emerging applications in theranostic nanomedicine.

In summary, the inventors have developed an enabling platform technology that offers extraordinary flexibility in tailoring the optical properties and structural diversity of plasmonic NNPs. The inventors have demonstrated that the adhesive and reactive nature of the PDA coating allows for rational designs of a broad spectrum of NNPs with customized combinations of functional cores and optically encoded nanogaps with desired gap sizes. The resulting multigap NNPs represent excellent model systems that support plasmon hybridization. More importantly, optically active multifunctional NNPs are of great potential in surface enhanced spectroscopy, biosensing, nanomedicine, and photocatalysis.

### EXAMPLE 21

### Synthesis of Au@PDA@Ag Nanomatryoshkas

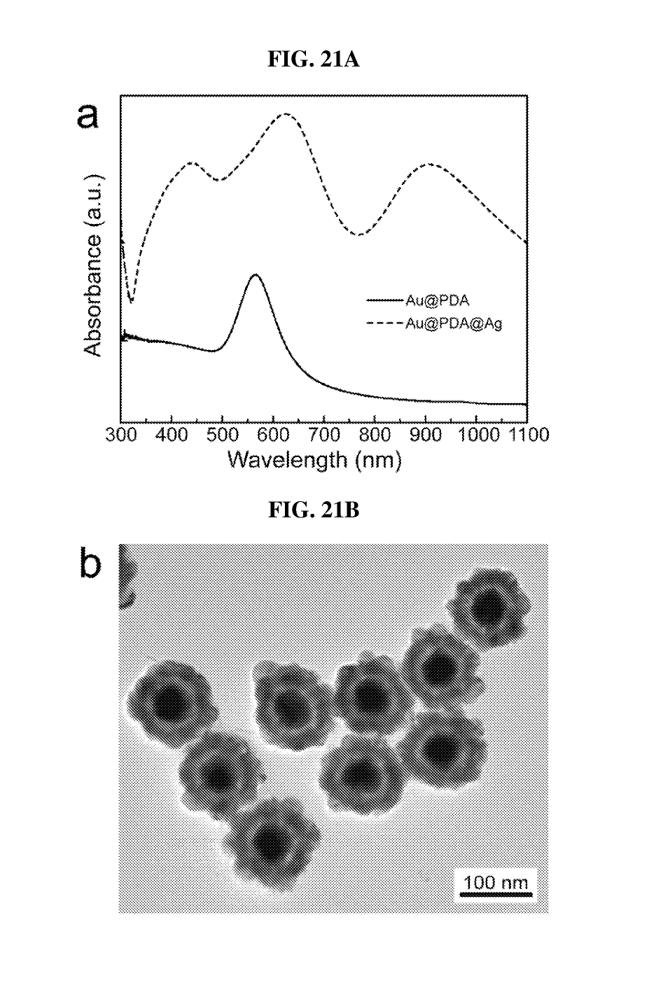

Typically, 80 μL of Au@PDA was added into 2 mL H₂O at 50° C. After stirring for 2 min, 75 μL of 2.5 mM AgNO₃ was injected, followed by 100 μL of 0.2 M NH₂OH.HCl. The color of the solution changed from light red to brown immediately. The reaction solution was stirred for 2 min. After cooling down, 50 μL of PEG-SH (10mg/mL) was added into the solution to further stabilize the nanomatryoshkas. Finally, the product was purified by centrifugation. FIG. 21A is a graph showing UV-vis spectra of Au@PDA and Au@PDA@Ag nanomatryoshkas, and FIG. 21B is TEM image of Au@PDA@Ag nanomatryoshkas.

### EXAMPLE 22

### Symmetry Breaking

The effect of symmetry breaking is an important research topic in the field of plasmonics. For plasmonic nanostructures which size is smaller than the wavelength of incident light, only plasmons with finite dipole moments can be excited. In the case of a symmetric nanoshell, symmetry breaking can be easily induced by a displacement of the dielectric core inside the metallic shell. This renders the plasmonic nanostructures higher-order multipolar modes dipole active and therefore visible in the optical spectrum of the nanoparticle. Meanwhile, much larger electromagnetic field enhancements can be produced in asymmetric nanostructures compared to their symmetric counterparts. Of particular interest, symmetry breaking results in Fano resonances caused by the interaction of narrow dark modes with broad bright modes. For strong interactions and near-degenerate levels, this coupling can lead to a plasmon-induced transparency of the nanostructure.

The facile strategy disclosed herein for nanogapped nanostructures with tailored core position enables a coupling between plasmon modes of differing multipolar order (FIGS. 22A and 22B), resulting in a tunable Fano resonance. It holds great potential for a wide range of applications, such as ultrasensitive media for chemical or biological analytical sensing, and new types of optical switches, interferometers, nanoantennas, and other devices.

### EXAMPLE 23

### Multi-Shell Nanogapped Nanoparticles

Raman molecules may be successfully located in the nanogaps with enhanced SERS signals. In addition, the completed shell avoids a possible signal fluctuation induced by desorption of Raman molecules or by the random aggregation-induced hot spots. Therefore, highly uniform SERS signals can be reproduced from each nanogapped nanostructure. Based on these merits, by increasing the number of shells and by changing the Raman molecules in different nanogaps, the Raman intensities and complex spectral profiles can be further modulated easily.

These multi-shell nanogapped nanoparticles with improved Raman signals and encoding capability may be easily designed and fabricated by the strategy disclosed herein. The resultant SERS probes open up new opportunities for multiplexed SERS-based biosensing and bioimaging.

### EXAMPLE 24

### Multifunctional Nanogapped Nanoparticles

The compatibility of a PDA coating with diverse core materials further allowed integrating a functional core with plasmonic nanogaps, achieving multifunctional NNPs. For example, imparting magnetic properties to NNPs by introducing a magnetic iron oxide core leads to magnetoplasmonic nanostructures of considerable interest for biosensing, theranostic, and catalytic applications. As a proof of concept, a new type of SERS-encoded magnetoplasmonic NNPs for the quantitative immunoassay of a common food-borne bacterial pathogen (i.e., E. coli O157:H7) was designed by combining magnetic separation and Raman spectroscopy for ultrasensitive detection and photothermal transduction (FIG. 17A).

Other functional cores, such as fluorescent quantum dots and mesoporous metal-organic frameworks, can also be introduced for a wider range of application.

### EXAMPLE 25

### Photothermal Therapy

Excited LSPR of plasmonic nanostructures releases energy through light scattering and heat dissipation. The photothermal conversion property of plasmonic nanostructures has made them compelling transducers for photothermal therapy (PTT) that is under intense research as a non-invasive therapeutic modality. The internal plasmonic coupling of NNPs shifts the LSPR into the NIR spectral range (FIGS. 8A and 8B), which is highly desirable for clinical translation because of a greater tissue penetration depth.

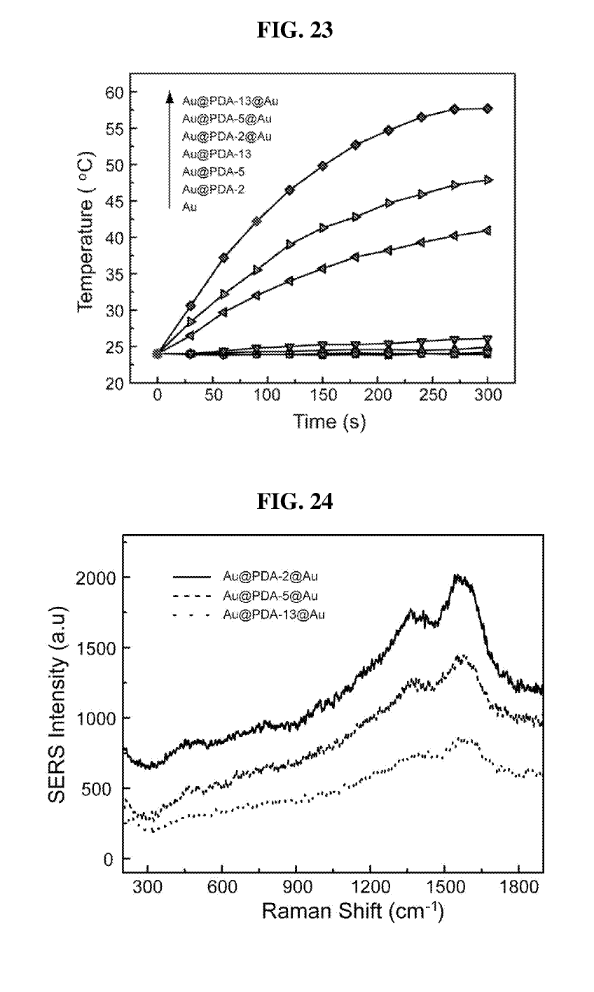

As shown in FIG. 23, the NNPs (20 nm core) of equal concentration (1.0 nM) all led to rapid temperature increase under an 808 nm laser excitation. The stronger absorption efficiency of the NNPs with larger gaps gave rise to better heating efficiency, reaching 47.9 and 57.7° C. for the NNPs with 5 and 13 nm nanogaps after 6 min irradiation. PDA-based nanoparticles recently have also been investigated as potential candidates for PTT. However, results obtained herein demonstrated that Au@PDA at the concentration used in this study only afforded a maximal temperature increase of 2.1° C., suggesting a better performance of plasmonic nanomaterials.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A core-shell plasmonic nanostructured material having a core and at least one shell surrounding the core, wherein the at least one shell comprises
a) a first layer comprising a polymer having a catechol group, the first layer defining a nanogap in the core-shell plasmonic nanostructured material, and
b) a second layer comprising a metal disposed on the first layer.

2. The core-shell plasmonic nanostructured material according to claim 1, wherein the polymer having a catechol group is selected from the group consisting of polydopamine, poly(norepinephrine), poly(L-3,4-dihydroxyphenylalanine), poly(5,6-dihydroxyl-1H-benzimidazole), polyphenol, dopamine-modified poly(L-glutamic acid), dopamine-modified polyphenol, dopamine-modified poly(ethyleneimine), polydopamine and Cu²⁺, polyphenol and Fe³⁺, copolymers thereof, and combinations thereof.

3. (canceled)

4. The core-shell plasmonic nanostructured material according to claim 1, wherein the first layer comprising a polymer having a catechol group is a continuous conformal coating of the polymer disposed on the core.

5. (canceled)

6. The core-shell plasmonic nanostructured material according to claim 1, wherein the first layer further comprises a signal probe.

7. The core-shell plasmonic nanostructured material according to claim 6, wherein the signal probe is at least one of a Raman probe or a fluorescent probe.

8.-9. (canceled)

10. The core-shell plasmonic nanostructured material according to claim 6, wherein the signal probe is covalently bonded to the polymer having a catechol group.

11. The core-shell plasmonic nanostructured material according to claim 1, wherein the metal comprised in the second layer is selected from the group consisting of gold, silver, copper, aluminum, platinum, palladium, and combinations thereof.

12. (canceled)

13. The core-shell plasmonic nanostructured material according to claim 1, wherein the second layer further comprises an analyte-binding molecule attached to the metal.

14. (canceled)

15. The core-shell plasmonic nanostructured material according to claim 1, wherein the core is a nanoparticle or a nanorod.

16. The core-shell plasmonic nanostructured material according claim 1, wherein the core comprises a material selected from the group consisting of a metal, a metal oxide, a metal-organic framework, a polymer, a magnetic material, a fluorescent quantum dot, and combinations thereof.

17. (canceled)

18. The core-shell plasmonic nanostructured material according to claim 1, wherein the core-shell plasmonic nanostructured material comprises two or more shells.

19. The core-shell plasmonic nanostructured material according to claim 18, wherein each first layer in the two or more shells has at least one of (i) a different polymer having a catechol group, (ii) a different thickness, or (iii) a different signal probe when present.

20. The core-shell plasmonic nanostructured material according to claim 18, wherein each second layer in the two or more shells comprises a different metal.

21. The core-shell plasmonic nanostructured material according to claim 1, wherein the at least one shell is concentrically or eccentrically disposed about the core.

22. (canceled)

23. A method for preparing a core-shell plasmonic nanostructured material having a core and at least one shell surrounding the core, the method comprising
a) providing a nanostructured material, and
b) forming at least one shell on the nanostructured material by
(i) forming a first layer comprising a polymer having a catechol group on the nanostructured material, the first layer defining a nanogap in the core-shell plasmonic nanostructured material, and
(ii) forming a second layer comprising a metal on the first layer.

24. The method according to claim 23, wherein forming the first layer is carried out by polymerizing monomers of the polymer having a catechol group on the nanostructured material.

25. The method according to claim 24, wherein forming the first layer further comprises covalently binding a signal probe to the polymer.

26.-27. (canceled)

28. The method according to claim 23, wherein the method comprises forming two or more shells on the nanostructured material.

29. The method according to claim 28, wherein forming each first layer of the two or more shells comprises forming each first layer using at least one of (i) a different polymer having a catechol group, (ii) a different thickness, or (iii) a different signal probe when present.

30. The method according to claim 28, wherein forming each second layer of the two or more shells comprises forming each second layer with a different metal.

31. (canceled)
